SEQUENCE LISTING (48 sequences)

SEQ ID NO: 1 - protein, 319 aa, synthetic construct
  REGION 1..319: note = WT-hemolysin
MKTRIVSSVTTTLLLGSILMNPVAGAADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 2 - protein, 27 aa, synthetic construct
  REGION 1..27: note = Signal Sequence
MKTRIVSSVTTTLLLGSILMNPVAGAA

SEQ ID NO: 3 - protein, 293 aa, synthetic construct
  REGION 1..293: note = Hemolysin without Signal Sequence
MDSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 4 - protein, 302 aa, synthetic construct
  REGION 1..302: note = hemolysin with Cysteine insert
MDSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDGGGGSCGGGGSKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 5 - protein, 361 aa, synthetic construct
  REGION 1..361: note = hemolysin with insert at position 129
  VARIANT 129..133: note = X can be any amino acid (linker residue)
  VARIANT 134..198: note = X can be any amino acid (linker residue) or absent
MDSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDXXXXXXXXXXXXXXXXXXXXXXXXXXXXXXXXXXXXXXXXXXXXXXXXXXXXXXXXXXXXXXXXXXXXXXKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 6 - protein, 293 aa, synthetic construct
  REGION 1..293: note = hemolysin with 48H -> L
MDSDINIKTGTTDIGSNTTVKTGDLVTYDKENGMHKKVFYSFIDDKNLNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 7 - protein, 361 aa, synthetic construct
  REGION 1..361: note = hemolysin with 48H -> L insert
  VARIANT 129..133: note = X can be any amino acid (linker residue)
  VARIANT 134..198: note = X can be any amino acid (linker residue) or absent
MDSDINIKTGTTDIGSNTTVKTGDLVTYDKENGMHKKVFYSFIDDKNLNKKLLVIRTKGTIAGQYRVYSEEGANKSGLAWPSAFKVQLQLPDNEVAQISDYYPRNSIDTKEYMSTLTYGFNGNVTGDDXXXXXXXXXXXXXXXXXXXXXXXXXXXXXXXXXXXXXXXXXXXXXXXXXXXXXXXXXXXXXXXXXXXXXXKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 8 - protein, 302 aa, synthetic construct
  REGION 1..302: note = 48H -> L Cysteine Insert
MDSDINIKTGTTDIGSNTTVKTGDLVTYDKENGMHKKVFYSFIDDKNLNKKLLVIRTKGTIAGQYRVYSEEGANKSGLAWPSAFKVQLQLPDNEVAQISDYYPRNSIDTKEYMSTLTYGFNGNVTGDDGGGGSCGGGGSKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 9 - protein, 271 aa, synthetic construct
  REGION 1..271: note = hemolysin with 2D-23G truncated amino acids 2D-23G are missing
MDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 10 - protein, 339 aa, synthetic construct
  REGION 1..339: note = hemolysin with 2D-23G truncated amino acids 2D-23G are missing with insert
  VARIANT 107..111: note = X can be any amino acid (linker residue)
  VARIANT 112..176: note = X can be any amino acid (linker residue) or absent
MDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDXXXXXXXXXXXXXXXXXXXXXXXXXXXXXXXXXXXXXXXXXXXXXXXXXXXXXXXXXXXXXXXXXXXXXXKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 11 - protein, 280 aa, synthetic construct
  REGION 1..280: note = hemolysin with 2D-23G truncated amino acids 2D-23G are missing with insert
MDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDGGGGSCGGGGSKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 12 - protein, 307 aa, synthetic construct
  REGION 1..307: note = hemolysin with L-6XHis-L
MDSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDGGGGSHHHHHHGGGGSKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 13 - protein, 317 aa, synthetic construct
  REGION 1..317: note = hemolysin with L2-6XHis-L2
MDSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDGGGGSGGGGSHHHHHHGGGGSGGGGSKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 14 - protein, 327 aa, synthetic construct
  REGION 1..327: note = hemolysin with L3-6XHis-L3
MDSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDGGGGSGGGGSGGGGSHHHHHHGGGGSGGGGSGGGGSKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 15 - protein, 315 aa, synthetic construct
  REGION 1..315: note = hemolysin with L-Somatostatin-L
MDSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDGGGGSAGCKNFFWKTFTSCGGGGSKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 16 - protein, 341 aa, synthetic construct
  REGION 1..341: note = hemolysin with L2-GLP1-L2
MDSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDGGGGSGGGGSHAEGTFTSDVSSYLEGQAAKEFIAWLVKGRGGGGSGGGGSKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 17 - protein, 308 aa, synthetic construct
  REGION 1..308: note = hemolysin with L-K3-L
MDSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDGGGGSKGGKGGKGGGGSKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 18 - protein, 308 aa, synthetic construct
  REGION 1..308: note = hemolysin with L-E3-L
MDSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDGGGGSEGGEGGEGGGGSKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 19 - protein, 376 aa, synthetic construct
  REGION 1..376: note = hemolysin with L2-Sac7e-L2
MDSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDGGGGSGGGGSMAKVRFKYKGEEKEVDTSKIKKVWRVGKMVSFTYDDNGKTGRGAVSEKDAPKELMDMLARAEKKKGGGGSGGGGSKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 20 - protein, 493 aa, synthetic construct
  REGION 1..493: note = hemolysin with L2-SNAP-L2
MDSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDGGGGSGGGGSMDKDCEMKRTTLDSPLGKLELSGCEQGLHRIIFLGKGTSAADAVEVPAPAAVLGGPEPLMQATAWLNAYFHQPEAIEEFPVPALHHPVFQQESFTRQVLWKLLKVVKFGEVISYSHLAALAGNPAATAAVKTALSGNPVPILIPCHRVVQGDLDVGGYEGGLAVKEWLLAHEGHRLGKPGLGGGGGSGGGGSKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 21 - protein, 5 aa, synthetic construct
  REGION 1..5: note = Linker 1 (L1)
GGGGS

SEQ ID NO: 22 - protein, 10 aa, synthetic construct
  REGION 1..10: note = Linker 2 (L2)
GGGGSGGGGS

SEQ ID NO: 23 - protein, 15 aa, synthetic construct
  REGION 1..15: note = Linker 3 (L3)
GGGGSGGGGSGGGGS

SEQ ID NO: 24 - protein, 16 aa, synthetic construct
  REGION 1..16: note = L1-6XHis-L1
GGGGSHHHHHHGGGGS

SEQ ID NO: 25 - protein, 26 aa, synthetic construct
  REGION 1..26: note = L2-6XHis-L2
GGGGSGGGGSHHHHHHGGGGSGGGGS

SEQ ID NO: 26 - protein, 36 aa, synthetic construct
  REGION 1..36: note = L3-6XHis-L3
GGGGSGGGGSGGGGSHHHHHHGGGGSGGGGSGGGGS

SEQ ID NO: 27 - protein, 24 aa, synthetic construct
  REGION 1..24: note = L1-Somatostatin-L1
GGGGSAGCKNFFWKTFTSCGGGGS

SEQ ID NO: 28 - protein, 50 aa, synthetic construct
  REGION 1..50: note = L2-GLP1-L2
GGGGSGGGGSHAEGTFTSDVSSYLEGQAAKEFIAWLVKGRGGGGSGGGGS

SEQ ID NO: 29 - protein, 17 aa, synthetic construct
  REGION 1..17: note = L1-K3-L1
GGGGSKGGKGGKGGGGS

SEQ ID NO: 30 - protein, 17 aa, synthetic construct
  REGION 1..17: note = L1-E3-L1
GGGGSEGGEGGEGGGGS

SEQ ID NO: 31 - protein, 85 aa, synthetic construct
  REGION 1..85: note = L2-Sac7e-L2
GGGGSGGGGSMAKVRFKYKGEEKEVDTSKIKKVWRVGKMVSFTYDDNGKTGRGAVSEKDAPKELMDMLARAEKKKGGGGSGGGGS

SEQ ID NO: 32 - protein, 202 aa, synthetic construct
  REGION 1..202: note = L2-SNAP-L2
GGGGSGGGGSMDKDCEMKRTTLDSPLGKLELSGCEQGLHRIIFLGKGTSAADAVEVPAPAAVLGGPEPLMQATAWLNAYFHQPEAIEEFPVPALHHPVFQQESFTRQVLWKLLKVVKFGEVISYSHLAALAGNPAATAAVKTALSGNPVPILIPCHRVVQGDLDVGGYEGGLAVKEWLLAHEGHRLGKPGLGGGGGSGGGGS

SEQ ID NO: 33 - protein, 292 aa, synthetic construct
  REGION 1..292: note = Alpha-Hemolysin without Signal Sequence and N-terminal Methionine
SDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDXXXKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 34 - protein, 126 aa, synthetic construct
  REGION 1..126: note = N-terminal domain of Alpha-Hemolysin
SDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDD

SEQ ID NO: 35 - protein, 105 aa, synthetic construct
  REGION 1..105: note = Truncated N-terminal domain of Alpha-Hemolysin
DLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDD

SEQ ID NO: 36 - protein, 162 aa, synthetic construct
  REGION 1..162: note = C-terminal domain of Alpha-Hemolysin
KIGGLIGANVSIGHTLKYQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 37 - protein, 20 aa, synthetic construct
  REGION 1..20: note = L-E2-L
GGGGSSSTSSDFRDRGGGGS

SEQ ID NO: 38 - protein, 41 aa, synthetic construct
  REGION 1..41: note = L-LZA-L
GGGGSAQLKKKLQALKKKNAQLKWKLQALKKKLAQKGGGGS

SEQ ID NO: 39 - protein, 41 aa, synthetic construct
  REGION 1..41: note = L-LZB-L
GGGGSAQLEKELQALEKENAQLEWELQALEKELAQKGGGGS

SEQ ID NO: 40 - protein, 34 aa, synthetic construct
  REGION 1..34: note = L-CPA-L
GGGGSGEIAALEKEIAALEWEIAALEQGSGGGGS

SEQ ID NO: 41 - protein, 34 aa, synthetic construct
  REGION 1..34: note = L-CPB-L
GGGGSGKIAALKYKIAALKKKIAALKQGSGGGGS

SEQ ID NO: 42 - protein, 23 aa, synthetic construct
  REGION 1..23: note = L-CPC-L
GGGGSEGGKGGEGGKGGEGGGGS

SEQ ID NO: 43 - protein, 23 aa, synthetic construct
  REGION 1..23: note = L-CPD-L
GGGGSKGGEGGKGGEGGKGGGGS

SEQ ID NO: 44 - protein, 75 aa, synthetic construct
  REGION 1..75: note = Sac7e
GGGGSMAKVRFKYKGEEKEVDTSKIKKVWRVGKMVSFTYDDNGKTGRGAVSEKDAPKELMDMLARAEKKKGGGGS

SEQ ID NO: 45 - protein, 192 aa, synthetic construct
  REGION 1..192: note = L-SNAP-L
GGGGSMDKDCEMKRTTLDSPLGKLELSGCEQGLHRIIFLGKGTSAADAVEVPAPAAVLGGPEPLMQATAWLNAYFHQPEAIEEFPVPALHHPVFQQESFTRQVLWKLLKVVKFGEVISYSHLAALAGNPAATAAVKTALSGNPVPILIPCHRVVQGDLDVGGYEGGLAVKEWLLAHEGHRLGKPGLGGGGGS

SEQ ID NO: 46 - protein, 248 aa, synthetic construct
  REGION 1..248: note = L-GFP-L
GGGGSMRKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGGGS

SEQ ID NO: 47 - protein, 419 aa, synthetic construct
  REGION 1..419: note = 130C-SNAP-GFP-Hemolysin with insert 1-CSNAP-GFP-1
MDKDCEMKRTTLDSPLGKLELSGCEQGLHRIIFLGKGTSAADAVEVPAPAAVLGGPEPLMQATAWLNAYFHQPEAIEEFPVPALHHPVFQQESFTRQVLWKLLKVVKFGEVISYSHLAALAGNPAATAAVKTALSGNPVPILIPCHRVVQGDLDVGGYEGGLAVKEWLLAHEGHRLGKPGLMRKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 48 - protein, 13 aa, synthetic construct
  SITE <1: note = maleimide
ATTCAACACTACC

(19) United States
(12) Patent Application Publication
(10) Pub. No.: US 2026/0125429 A1
(43) Pub. Date: May 7, 2026

(54) MEMBRANE TRANSLOCATION COMPOSITIONS AND METHODS

(71) Applicant: THE REGENTS OF THE UNIVERSITY OF CALIFORNIA, Oakland, CA (US)

(72) Inventors: Alexander HARJUNG, La Jolla, CA (US); Neal DEVARAJ, San Diego, CA (US)

(21) Appl. No.: 19/291,412

(22) Filed: Aug. 5, 2025

Related U.S. Application Data
(63) Continuation of application No. PCT/US2024/015069, filed on Feb. 8, 2024.
(60) Provisional application No. 63/483,836, filed on Feb. 8, 2023.

(51) Int. Cl. C07K 14/195

(52) U.S. Cl. CPC C07K 14/195; C07K 2319/00

(57) ABSTRACT

Provided herein, inter alia, are compositions and methods relating to α-Hemolysin polypeptide variants for translocation across phospholipid membranes.

### CROSS-REFERENCE TO RELATED APPLICATIONS

The present application is a continuation of International Application No. PCT/US2024/015069, filed Feb. 8, 2024, which claims priority under 35 U.S.C. § 119(e) to U.S. Provisional Patent Application No. 63/483,836, filed Feb. 8, 2023, each which are hereby incorporated by reference in its entirety.

### STATEMENT AS TO RIGHTS TO INVENTIONS MADE UNDER FEDERALLY SPONSORED RESEARCH AND DEVELOPMENT

This invention was made with government support under N00014-22-1-2800 awarded by the Office of Naval Research. The government has certain rights in the invention.

### INCORPORATION BY REFERENCE OF SEQUENCE LISTING

The instant application contains a Sequence Listing submitted as an electronic .xml file named 2024-06-06 Sequence_Listing_ST26 048537-657001WO.xml created Jun. 6, 2024 and is 71,628 bytes in size.

### BACKGROUND

Functionalization of the cell membrane with cell-surface proteins and receptors is a requirement for many cellular functions. It enables communication and interaction with the extracellular environment and plays a key role in various cellular processes such as cell-cell adhesion. Natural cells use transmembrane proteins to functionalize the cell membrane. They have therefore evolved different complex mechanisms for the insertion of proteins into cell membranes. Since these highly complex pathways are dependent on multiple proteins and cofactors, reconstituting them into artificial cells would be extremely challenging. For this reason, artificial systems are mostly limited to reconstituting membrane proteins through detergent-based methods, which are often low-yielding and non-trivial to successfully perform on complex transmembrane protein systems. Likewise, it is a common practice to functionalize preformed vesicles with proteins or peptides from the outside, by anchoring them to the membrane. However, none of these methods resemble the natural process in which cells express a protein in the cytoplasm and then insert the protein into the cell membrane.

Pore forming toxins (PFTs) can self-insert into biological membranes, independent of any insertion machinery. α-hemolysin (αHL) is an example of a PFT. Pore formation occurs when αHL binds to the lipid membrane, first as a soluble monomer, and then subsequently forming a heptameric complex, which spontaneously translocates across the lipid membrane as a barrel-like structure. αHL has been extensively studied due to its biological role in bacterial infection and its utility in nanopore sequencing. Due to its self-insertion and pore-forming ability, αHL has also been used in artificial cell systems to make lipid membranes permeable to small molecules, which can promote internal biochemical reactions like transcription and translation.

### BRIEF SUMMARY

Each of the aspects and embodiments described herein are capable of being used together, unless excluded either explicitly or clearly from the context of the embodiment or aspect.

In an aspect is provided an α-Hemolysin polypeptide comprising an amino acid insert sequence between amino acids corresponding to positions D128 and K131 of SEQ ID NO:3, wherein the amino acid insert sequence is 5 to 70 amino acids in length.

In embodiments, the α-Hemolysin polypeptide without said amino acid insert sequence has at least 80%, 85%, 90%, 91%, 92%, 93%, 94%, 95%, 96%, 97%, 98%, or 99% sequence identity to SEQ ID NO: 3. In embodiments, the amino acid sequence between amino acids corresponding to D128 and K131 of SEQ ID NO:3 comprises an amino acid deletion. In embodiments, the α-Hemolysin polypeptide comprises a cysteine mutation corresponding to a position of amino acid 130 of SEQ ID NO:3. In embodiments, the α-Hemolysin polypeptide comprises a histidine to leucine mutation. In embodiments, the α-Hemolysin polypeptide comprises a deletion corresponding to amino acids D2-G23 of SEQ ID NO:3. In embodiments, the amino acid insert sequence is less than 41 amino acids in length. In embodiments, the amino acid insert sequence is less than 52 amino acids in length. In embodiments, the amino acid insert sequence comprises a cysteine. In embodiments, the amino acid insert sequence comprises a glycine linker. In embodiments, the α-Hemolysin additionally comprises a bioconjugate reactive moiety. In embodiments, the bioconjugate reactive moiety is attached to an amino acid of the amino acid insert sequence. In embodiments, the α-Hemolysin additionally comprises a cargo molecule. In embodiments, the cargo molecule is attached to the amino acid insert sequence. In embodiments, the cargo molecule is covalently attached to a cysteine in the amino acid insert sequence. In embodiments, the cargo molecule is covalently attached by a bioconjugate to a cysteine in the amino acid insert sequence. In embodiments, the cargo molecule is selected from the group of a polynucleotide, a polypeptide, a lipid, a carbohydrate, or a small molecule.

In an aspect is provided a phospholipid membrane comprising an α-Hemolysin polypeptide of the disclosure.

In embodiments, the phospholipid further comprises a macromolecule. In embodiments, the macromolecule is a polypeptide or polynucleotide. In embodiments, the polynucleotide is DNA or RNA. In embodiments, the polynucleotide is mRNA, siRNA, miRNA, or microRNA. In embodiments, the macromolecule comprises a bioconjugate reactive moiety. In embodiments, the bioconjugate reactive moiety is a maleimide. In embodiments, the macromolecule comprises a bioconjugate.

In an aspect is provided a tissue-like structure comprising a plurality of phospholipid membranes of the disclosure.

In an aspect is provided a peptide of the formula: AA¹-PS¹-L1-PS²-AA² wherein PS1 is an amino acid sequence having at least 90% sequence identity to SEQ ID: 35; PS² is an amino acid sequence having at least 90% sequence identity to SEQ ID: 36; L¹ is a peptidyl linker having 1-70 amino acids; and AA¹ and AA² are independently hydrogen or amino acid sequences having 1-100 amino acids.

In embodiments, the PS¹ is SEQ ID: 34. In embodiments, the PS¹ comprises a Histidine to Leucine mutation corresponding to a position of amino acid H25 of SEQ ID NO:35. In embodiments, the L¹ is selected from the amino acid sequences in Table 2. In embodiments, the L¹ comprises a cysteine residue. In embodiments, the L¹ is less than 41 amino acids in length. In embodiments, the L¹ is less than 52 amino acids in length. In embodiments, the L¹ comprises a glycine linker. In embodiments, AA¹ and/or AA² are a fusion protein. In embodiments, the peptide additionally comprises a bioconjugate reactive moiety. In embodiments, the bioconjugate reactive moiety is attached to an amino acid in L¹. In embodiments, the peptide additionally comprises a cargo molecule. In embodiments, the cargo molecule is attached to L. In embodiments, the cargo molecule is covalently attached to a cysteine in L¹. In embodiments, the cargo molecule is covalently attached by a bioconjugate to a cysteine in L¹. In embodiments, the cargo molecule is selected from the group of a polynucleotide, a polypeptide, a lipid, a carbohydrate, or a small molecule.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A shows a general scheme of Hemolysin (HL)-based peptide translocation. A peptide is inserted between amino acids D128 and K131 of the WT-α-HL sequence. The hemolysin self-translocated and assembles as a pore forming hexamer in a membrane. The inserted peptide is translocated across the membrane during pore assembly. FIG. 1B shows Cy5-encapsulating GUV 5 minutes after treatment with αHL-130His6-GFP, showing membrane binding of the Protein. FIG. 1C shows Cy5-encapsulating GUV 60 minutes after treatment with αHL-130His6-GFP showing full loss of the Cy5 signal due to leakage.

FIG. 2A shows a general scheme for the antibody assay binding assay. Cy5-conjugated anti-His-tag antibody is encapsulated into GUVs. Upon treatment with αHL-130His6-GFP, the antibody is localized to the membrane, indicating that the His-tag of the αHL-130His6-GFP was fully translocated and can be bound by the antibody. FIG. 2B shows anti-His-tag antibody encapsulating GUV 60 min after treatment with αHL-130His6-GFP. Membrane shows strong Cy5 (antibody) signal. FIG. 2C shows untreated anti His-tag antibody in the encapsulating GUV after 60 min. Without treatment with αHL-130His6-GFP, the antibody does not bind to the membrane.

FIG. 3A shows a general scheme for testing translocation of peptides by GUV antibody binding assay. A Cy5-conjugated monoclonal antibody specific for given peptide insert is encapsulated into GUVs. Upon external treatment of GUVs with αHL-GFP fusion proteins containing the peptide insert, the antibody will localize to the membrane, indicating that the peptide insert has fully translocated across the membrane and can be bound by the internal antibody. FIG. 3B shows GUVs encapsulated with Cy5 modified anti-6×His-tag antibodies before and after treatment with αHL-GFP containing the L-6×His-L loop insert. The GUV membrane shows increased Cy5 (antibody) signal after treatment. FIG. 3C shows a larger population of GUVs encapsulating the Cy5 anti-6×His-tag antibody, all showing increased membrane fluorescence after treatment with αHL-GFP containing the L-6×His-L loop insert. FIG. 3D shows Cy5 antibody assays with all the peptide inserts that were shown to form functional pores. Scale bar: 10 μm.

FIG. 4A shows a general scheme for artificial tissue formation. Hemolysin protein is expressed inside GUVs. Membrane insertion leads to translocation of the His-tag which is then able to interact with a second population of GUVs (dark grey) doped with a NiNTA-lipid. FIG. 4B shows assembled tissue-like structure. Light grey vesicles are functionalized by internal expression of αHL-130His6. Dark grey vesicles are doped with NiNTA. In order to differentiate the populations using fluorescence microscopy, the light grey, αHL-expressing GUVs also contained FITC-Dextran, while the dark grey NiNTA GUVs were doped with 0.01% PE Topfluor AF594. The interaction between the two populations of GUVs seems to be so strong that the GUV were compressed leading to a significant shape change.

FIG. 5A shows αHL pores with the K3 peptide insert can interact with pores containing the E3 peptide insert due to electrostatic interactions. FIG. 5B shows a general scheme of the tissue formation experiment. Two populations of GUVs express αHL with one of two mutually interacting peptide inserts, K3 (positively charged) or E3 (negatively charged). After internal protein expression, αHL oligomerizes on the artificial membrane and forms pores, while at the same time translocating the K3 or E3 peptide insert across the membrane for extracellular display. After translocation, the K3 expressing GUVs can interact with E3 expressing GUVs leading to GUV self-assembly into tissue-like structures. FIG. 5C shows confocal microscopy images of tissue-like structures formed through mixing K3-αHL and E3-αHL expressing GUVs. Specific interactions between K3-αHL and E3-αHL expressing vesicles were observed and controls (see supporting information) did not result in the formation of assemblies. Scale bar: 10 μm.

FIG. 6A shows a general scheme of an artificial cell signaling pathway using hydrogen peroxide. Glucose oxidase (GO) containing sender artificial cells (marked with mCherry) create hydrogen peroxide in the presence of glucose. Hydrogen peroxide can enter receiver artificial cells which contain Hyper7 as a peroxide reporter. Catalase (CA) is added to the extracellular solution to prevent peroxide signaling outside of the artificial tissue-like structures. FIG. 6B shows a confocal microscopy image of hydrogen peroxide signaling in a large tissue-like assembly. Hyper7 containing artificial cells inside the vesicular assembly show increased fluorescence in the green channel. Hyper 7 containing artificial cells outside of tissue (marked with white boxes) show much weaker fluorescence signal. Scale bar: 25 μm. FIG. 6C shows the quantification of Hyper7 fluorescence intensity. 1: Control experiment. Mean fluorescence of artificial cells in tissue-like structure in the absence of glucose. 2: Mean fluorescence of artificial cells outside of tissue-like structure in the presence of glucose. 3: Mean fluorescence of artificial cells in the tissue-like structure in the presence of glucose.

FIG. 7A shows a general scheme for oligo hybridization assay. DNA-oligo is conjugated to αHL-130C. Complementary oligo labeled with FITC is encapsulated into GUVs. Upon treatment with conjugate, the oligos hybridize and the FITC-signal is localized to the membrane. FIG. 7B shows a protein gel: Conjugation of oligo to αHL-130C leads to gel shift. FIG. 7C shows translocation of αHL-130C conjugated oligo leads to hybridization with FITC labeled complementary oligo which gets localized to GUV membrane.

FIG. 8 shows SNAP-GFP translocation through GUV-membrane by 130C-Hemolysin. Green Channel: Green fluorescence signal indicates that SNAP-tagged protein was inserted into the membrane. Red Channel: Protein insertion leads to leakage of Cy5, indicating formation of fully functional pores und full translocation of the SNAP-GFP insert.

FIG. 9A and FIG. 9B show FITC fluorescence in THP1 cells after incubation with αHL attached to a FITC labeled oligo after 5 min (FIG. 9A) and 30 min (FIG. 9B).

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

The terms “bind” and “bound” as used herein is used in accordance with its plain and ordinary meaning and refers to the association between atoms or molecules. The association can be direct or indirect. For example, bound atoms or molecules may be bound, e.g., by covalent bond, linker (e.g., a first linker or second linker), or non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like).

As used herein, the term “conjugated” when referring to two moieties means the two moieties are bonded, wherein the bond or bonds connecting the two moieties may be covalent or non-covalent. In embodiments, the two moieties are covalently bonded to each other (e.g., directly or through a covalently bonded intermediary). In embodiments, the two moieties are non-covalently bonded (e.g., through ionic bond(s), van der Waal's bond(s)/interactions, hydrogen bond(s), polar bond(s), or combinations or mixtures thereof).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature. In embodiments, an amino acid can be a protein-bound amino acid (e.g., part of a peptide or protein) or a free amino acid (e.g., not part of peptide or protein). In embodiments, a free amino acid is exogenously administered.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide,” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see. e.g., NCBI web site: the world wide web (www) at ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. For example, amino acids corresponding to positions D128 and K131 of SEQ ID NO:3 would be the following emboldened and underlined amino acids in SEQ ID NO:1:

MKTRIVSSVTTTLLLGSILMNPVAGAADSDINIKTGTTDIGSNTTVKT
GDLVTYDKENGMHKKVFYSFIDDKNHNKKLLVIRTKGTIAGQYRVYSE
EGANKSGLAWPSAFKVQLQLPDNEVAQISDYYPRNSIDTKEYMSTLTY
GFNGNVTGDDTGKIGGLIGANVSIGHTLKYVQPDFKTILESPTDKKVG
WKVIFNNMVNQNWGPYDRDSWNPVYGNQLFMKTRNGSMKAADNFLDPN
KASSLLSSGFSPDFATVITMDRKASKQQTNIDVIYERVRDDYQLHWTS
TNWKGTNTKDKWTDRSSERYKIDWEKEEMTN.

An amino acid residue in a protein “corresponds” to a given residue when it occupies the same essential structural position within the protein as the given residue.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

As used herein, the term “vesicles” or “lipid vesicles” is used in accordance with its plain ordinary meaning and refers to a structure within or outside a cell, consisting of liquid or cytoplasm enclosed by a lipid bilayer. The vesicles form naturally during the processes of secretion (exocytosis), uptake (endocytosis) and transport of materials within the plasma membrane. Alternatively, they may be prepared artificially, in which case they are called liposomes (not to be confused with lysosomes). If there is only one phospholipid bilayer, they are called unilamellar liposome vesicles; otherwise they are called multilamellar. The membrane enclosing the vesicle is also a lamellar phase, similar to that of the plasma membrane, and intracellular vesicles may fuse with the plasma membrane to release their contents outside the cell. The vesicles may also fuse with other organelles within the cell. A vesicle released from the cell is known as an extracellular vesicle. The vesicles perform a variety of functions. Because it is separated from the cytosol, the inside of the vesicle may be made to be different from the cytosolic environment. For this reason, the vesicles are a basic tool used by the cell for organizing cellular substances. The vesicles are involved in metabolism, transport, buoyancy control, and temporary storage of food and enzymes. The vesicles may also act as chemical reaction chambers.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may include natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “aptamer” as provided herein refers to oligonucleotides (e.g., short oligonucleotides or deoxyribonucleotides), that bind (e.g., with high affinity and specificity) to proteins, peptides, and small molecules. Aptamers typically have defined secondary or tertiary structure owing to their propensity to form complementary base pairs and, thus, are often able to fold into diverse and intricate molecular structures. The three-dimensional structures are essential for aptamer binding affinity and specificity, and specific three-dimensional interactions drives the formation of aptamer-target complexes. Aptamers can be selected in vitro from very large libraries of randomized sequences by the process of systemic evolution of ligands by exponential enrichment (SELEX as described in Ellington A D, Szostak J W (1990) In vitro selection of RNA molecules that bind specific ligands. Nature 346:818-822; Tuerk C, Gold L (1990) Systematic evolution of ligands by exponential enrichment: RNA ligands to bacteriophage T4 DNA polymerase. Science 249:505-510) or by developing SOMAmers (slow off-rate modified aptamers) (Gold L et al. (2010) Aptamer-based multiplexed proteomic technology for biomarker discovery. PLoS ONE 5(12):e15004). Applying the SELEX and the SOMAmer technology includes for instance adding functional groups that mimic amino acid side chains to expand the aptamer's chemical diversity. As a result high affinity aptamers for almost any protein target are enriched and identified. Aptamers exhibit many desirable properties for targeted drug delivery, such as ease of selection and synthesis, high binding affinity and specificity, flexible structure, low immunogenicity, and versatile synthetic accessibility.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The term “biomolecule” refers to an organic molecule, which is involved in the maintenance and metabolic processes of a living organisms. Biomolecules include for example small molecules such as primary and secondary metabolites, vitamins, and hormones, and macromolecules such as peptides, proteins, nucleic acids, carbohydrates, and lipids.

The term “small molecule” refers to organic molecules with a molecular weight of less than 1000 Da. Small molecules include, but are not limited to inhibitors, activators, and antivirals.

The term “drug” refers to a therapeutic molecule that is administered to a subject in need thereof. Drugs include but are not limited to small molecule drugs and biomolecules. Biomolecules can be small molecules, peptides, antibodies, proteins, prodrugs, and nucleic acids.

A “cell” as used herein, refers to a cell carrying out metabolic or other functions sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The term “plasmid,” “expression vector,” or “viral vector” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids. Suitable viral vectors contemplated herein include, for example, lentiviral vectors and onco-retroviral vectors.

As used herein, the term “expression” is used in accordance with its plain ordinary meaning and refers to a step involved in the production of the polypeptide including, but not limited to, transcription, post-transcriptional modification, translation, post-translational modification, and secretion. Expression may be detected using conventional techniques for detecting protein (e.g., ELISA, Western blotting, flow cytometry, immunofluorescence, immunohistochemistry, etc.).

### II. Pore Forming Toxins

Provided herein are pore forming toxins (e.g., a beta barrel pore forming toxin) with a modification in the intracellular loop. In embodiments, the pore forming toxin includes an amino acid sequence between secondary structural elements of the pore forming toxin (e.g., a beta barrel or alpha helix) referred to herein as a “loop.” In embodiments, where the pore forming toxin forms a pore within the lipid bilayer membrane of a cell, the loop resides in the intracellular space. In embodiments, the pore forming toxin comprises an amino acid insert sequence within or replacing (either partially or fully) the amino acid sequence of the loop, wherein the amino acid insert sequence insert is 1 to 75 amino acids.

In an aspect, the pore forming toxin is an α-Hemolysin (αHL).

In an aspect is provided a α-Hemolysin with a modification in the loop. In embodiments, the modification is an amino acid insert sequence within or replacing (either partially or fully) the amino acid sequence of the loop, wherein the amino acid insert sequence is 1 to 75 amino acids in length.

α-Hemolysin (αHL) is a β-barrel pore-forming toxin (βPFT), that includes two domains: the extracellular domain and the pore forming transmembrane domain. The transmembrane domain may be formed by two beta strands connected by a loop region. Monomers of α-Hemolysin can self-insert into lipid bilayers resulting in the formation of pores comprised of an α-Hemolysin heptamer. Upon pore formation, the loop region (i.e., intracellular loop or loop) between the two beta strands may be located on the opposite lipid bilayer side compared to the extracellular domain.

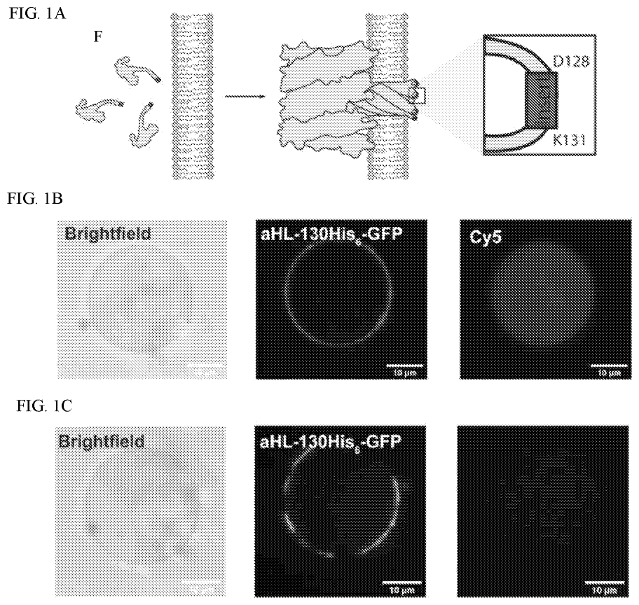

In embodiments, α-Hemolysin (αHL) or alpha toxin (e.g., GenBank: CAA25801.1, UNIPROT: P09616), is a major cytotoxic agent released by the bacterium Staphylococcus aureus and the first identified member of the pore forming beta-barrel toxin family. α-Hemolysin can self-insert into lipid bilayers resulting in the formation of pores comprised of an α-Hemolysin heptamer. αHL can be mutated in several positions without losing its pore forming activity. While αHL assembly may lead to spontaneous formation of a multimeric transmembrane protein, the loop formed by the amino acids corresponding to amino acids 154-157 of SEQ ID NO: 1 or 128-131 of SEQ ID NO:3 may fully translocate across the lipid bilayer upon pore formation (FIG. 1A).

α-Hemolysin without signal sequence with N-terminal Methionine,
SEQ ID NO: 3:
MDSDINIKTGTTDIGSNTTVKTGDLVTYDKENGMHKKVFYSFIDDKNHNK
1 10 20 30 40 50
KLLVIRTKGTIAGQYRVYSEEGANKSGLAWPSAFKVQLQLPDNEVAQISD
51 60 70 80 90 100
YYPRNSIDTKEYMSTLTYGFNGNVTGDDTGKIGGLIGANVSIGHTLKYVQ
101 110 120 130 140 150
PDFKTILESPTDKKVGWKVIFNNMVNQNWGPYDRDSWNPVYGNQLFMKTR
151 160 170 180 190 200
NGSMKAADNFLDPNKASSLLSSGFSPDFATVITMDRKASKQQTNIDVIYE
201 210 220 230 240 250
RVRDDYQLHWTSTNWKGTNTKDKWTDRSSERYKIDWEKEEMTN
251 260 270 280 290

In one aspect, the α-Hemolysin is a wild type (WT) α-Hemolysin, wherein the α-Hemolysin comprises an amino acid insert sequence between the residues corresponding to residues D154 and K157 of SEQ ID NO: 1, wherein the amino acid insert sequence is from 1-70 amino acids in length. In one aspect, the α-Hemolysin is a WT α-Hemolysin without the signal sequence.

In all embodiments, where an amino acid insert sequence is stated to be “between” recited residues in specified sequence (e.g., between the residues corresponding to residues D154 and K157 of SEQ ID NO: 1), the amino acid insert sequence completely replaces the amino acids in the specified sequence between the recited residues. For example, in embodiments, where the amino acid insert sequence is between the residues corresponding to residues D154 and K157 of SEQ ID NO: 1, the amino acid insert sequence replaces the amino acids at position 155 and 156 in SEQ ID NO: 1. In embodiments, where an amino acid insert sequence is stated to be “between” recited residues in specified sequence (e.g., between the residues corresponding to residues D154 and K157 of SEQ ID NO: 1), the amino acid insert sequence partially replaces the amino acids in the specified sequence between the recited residues. For example, in embodiments, where the amino acid insert sequence is between the residues corresponding to residues D154 and K157 of SEQ ID NO: 1, the amino acid insert sequence replaces the amino acids at position 155 in SEQ ID NO: 1 but the amino acid at position 156 in SEQ ID NO: 1 remains present. In embodiments, where an amino acid insert sequence is stated to be “between” recited residues in specified sequence (e.g., between the residues corresponding to residues D154 and K157 of SEQ ID NO: 1), the amino acid insert sequence does not replace any of the amino acids in the specified sequence between the recited residues. For example, in embodiments, where the amino acid insert sequence is between the residues corresponding to residues D154 and K157 of SEQ ID NO: 1, the amino acid insert sequence the amino acids at position 155 in SEQ ID NO: 1 and the amino acid at position 156 in SEQ ID NO: 1 remain present.

In one aspect, the α-Hemolysin comprises an amino acid sequence selected from Table 1. In one aspect, the α-Hemolysin is selected from SEQ ID NO: 1 or 3-11. In one aspect, the α-Hemolysin is an α-Hemolysin having at least 80%, 85%, 90%, 91%, 92%, 93%, 94%, 95%, 96%, 97%, 98%, or 99% sequence identity to SEQ ID NO: 1, wherein the α-Hemolysin comprises an amino acid insert sequence between D154 and K157 wherein the amino acid insert sequence is from 1-70 amino acids in length. In one aspect, the α-Hemolysin is a α-Hemolysin having at least 80% sequence identity to SEQ ID NO: 1, wherein the α-Hemolysin comprises an amino acid insert sequence between D154 and K157 wherein the amino acid insert sequence is from 1-70 amino acids in length. In one aspect, the α-Hemolysin is a α-Hemolysin having at least 85% sequence identity to SEQ ID NO: 1, wherein the α-Hemolysin comprises an amino acid insert sequence between D154 and K157 wherein the amino acid insert sequence is from 1-70 amino acids in length. In one aspect, the α-Hemolysin is a α-Hemolysin having at least 90% sequence identity to SEQ ID NO: 1, wherein the α-Hemolysin comprises an amino acid insert sequence between D154 and K157 wherein the amino acid insert sequence is from 1-70 amino acids in length. In one aspect, the α-Hemolysin is a α-Hemolysin having at least 91% sequence identity to SEQ ID NO: 1, wherein the α-Hemolysin comprises an amino acid insert sequence between D154 and K157 wherein the amino acid insert sequence is from 1-70 amino acids in length. In one aspect, the α-Hemolysin is a α-Hemolysin having at least 92% sequence identity to SEQ ID NO: 1, wherein the α-Hemolysin comprises an amino acid insert sequence between D154 and K157 wherein the amino acid insert sequence is from 1-70 amino acids in length. In one aspect, the α-Hemolysin is a α-Hemolysin having at least 93% sequence identity to SEQ ID NO: 1, wherein the α-Hemolysin comprises an amino acid insert sequence between D154 and K157 wherein the amino acid insert sequence is from 1-70 amino acids in length. In one aspect, the α-Hemolysin is a α-Hemolysin having at least 94% sequence identity to SEQ ID NO: 1, wherein the α-Hemolysin comprises an amino acid insert sequence between D154 and K157 wherein the amino acid insert sequence is from 1-70 amino acids in length. In one aspect, the α-Hemolysin is a α-Hemolysin having at least 95% sequence identity to SEQ ID NO: 1, wherein the α-Hemolysin comprises an amino acid insert sequence between D154 and K157 wherein the amino acid insert sequence is from 1-70 amino acids in length. In one aspect, the α-Hemolysin is a α-Hemolysin having at least 96% sequence identity to SEQ ID NO: 1, wherein the α-Hemolysin comprises an amino acid insert sequence between D154 and K157 wherein the amino acid insert sequence is from 1-70 amino acids in length. In one aspect, the α-Hemolysin is a α-Hemolysin having at least 97% sequence identity to SEQ ID NO: 1, wherein the α-Hemolysin comprises an amino acid insert sequence between D154 and K157 wherein the amino acid insert sequence is from 1-70 amino acids in length. In one aspect, the α-Hemolysin is a α-Hemolysin having at least 98% sequence identity to SEQ ID NO: 1, wherein the α-Hemolysin comprises an amino acid insert sequence between D154 and K157 wherein the amino acid insert sequence is from 1-70 amino acids in length. In one aspect, the α-Hemolysin is a α-Hemolysin having at least 99% sequence identity to SEQ ID NO: 1, wherein the α-Hemolysin comprises an amino acid insert sequence between D154 and K157 wherein the amino acid insert sequence is from 1-70 amino acids in length.

In one aspect, the α-Hemolysin is a α-Hemolysin having at least 80%, 85%, 90%, 91%, 92%, 93%, 94%, 95%, 96%, 97%, 98%, or 99% sequence identity to a 200 amino acid continuous sequence within SEQ ID NO: 1 wherein the α-Hemolysin comprises an amino acid insert sequence between D154 and K157 wherein the amino acid insert sequence is from 1-70 amino acids in length. In one aspect, the α-Hemolysin is a α-Hemolysin having at least 90% sequence identity to a 200 amino acid continuous sequence within SEQ ID NO: 1 wherein the α-Hemolysin comprises an amino acid insert sequence between D154 and K157 wherein the amino acid insert sequence is from 1-70 amino acids in length. In one aspect, the α-Hemolysin is a α-Hemolysin having at least 95% sequence identity to a 200 amino acid continuous sequence within SEQ ID NO: 1 wherein the α-Hemolysin comprises an amino acid insert sequence between D154 and K157 wherein the amino acid insert sequence is from 1-70 amino acids in length. In one aspect, the α-Hemolysin is a α-Hemolysin having at least 98% sequence identity to a 200 amino acid continuous sequence within SEQ ID NO: 1 wherein the α-Hemolysin comprises an amino acid insert sequence between D154 and K157 wherein the amino acid insert sequence is from 1-70 amino acids in length. In one aspect, the α-Hemolysin is a α-Hemolysin having at least 80%, 85%, 90%, 91%, 92%, 93%, 94%, 95%, 96%, 97%, 98%, or 99% sequence identity to a 200 amino acid continuous sequence within SEQ ID NO: 1 wherein the α-Hemolysin comprises an amino acid insert sequence between D154 and K157 wherein the amino acid insert sequence is from 1-55 amino acids in length. In one aspect, the α-Hemolysin is a α-Hemolysin having at least 90% sequence identity to a 200 amino acid continuous sequence within SEQ ID NO: 1 wherein the α-Hemolysin comprises an amino acid insert sequence between D154 and K157 wherein the amino acid insert sequence is from 1-55 amino acids in length. In one aspect, the α-Hemolysin is a α-Hemolysin having at least 95% sequence identity to a 200 amino acid continuous sequence within SEQ ID NO: 1 wherein the α-Hemolysin comprises an amino acid insert sequence between D154 and K157 wherein the amino acid insert sequence is from 1-55 amino acids in length. In one aspect, the α-Hemolysin is a α-Hemolysin having at least 98% sequence identity to a 200 amino acid continuous sequence within SEQ ID NO: 1 wherein the α-Hemolysin comprises an amino acid insert sequence between D154 and K157 wherein the amino acid insert sequence is from 1-55 amino acids in length.

In one aspect, the α-Hemolysin is a α-Hemolysin having at least 80%, 85%, 90%, 91%, 92%, 93%, 94%, 95%, 96%, 97%, 98%, or 99% sequence identity to SEQ ID NO: 3, wherein the α-Hemolysin comprises an amino acid insert sequence between D128 and K131 wherein the amino acid insert sequence is from 1-70 amino acids in length. In one aspect, the α-Hemolysin is a α-Hemolysin having at least 80% sequence identity to SEQ ID NO: 3, wherein the α-Hemolysin comprises an amino acid insert sequence between D128 and K131 wherein the amino acid insert sequence is from 1-70 amino acids in length. In one aspect, the α-Hemolysin is a α-Hemolysin having at least 85% sequence identity to SEQ ID NO: 3, wherein the α-Hemolysin comprises an amino acid insert sequence between D128 and K131 wherein the amino acid insert sequence is from 1-70 amino acids in length. In one aspect, the α-Hemolysin is a α-Hemolysin having at least 90% sequence identity to SEQ ID NO: 3, wherein the α-Hemolysin comprises an amino acid insert sequence between D128 and K131 wherein the amino acid insert sequence is from 1-70 amino acids in length. In one aspect, the α-Hemolysin is a α-Hemolysin having at least 91% sequence identity to SEQ ID NO: 3, wherein the α-Hemolysin comprises an amino acid insert sequence between D128 and K131 wherein the amino acid insert sequence is from 1-70 amino acids in length. In one aspect, the α-Hemolysin is a α-Hemolysin having at least 92% sequence identity to SEQ ID NO: 3, wherein the α-Hemolysin comprises an amino acid insert sequence between D128 and K131 wherein the amino acid insert sequence is from 1-70 amino acids in length. In one aspect, the α-Hemolysin is a α-Hemolysin having at least 93% sequence identity to SEQ ID NO: 3, wherein the α-Hemolysin comprises an amino acid insert sequence between D128 and K131 wherein the amino acid insert sequence is from 1-70 amino acids in length. In one aspect, the α-Hemolysin is a α-Hemolysin having at least 94% sequence identity to SEQ ID NO: 3, wherein the α-Hemolysin comprises an amino acid insert sequence between D128 and K131 wherein the amino acid insert sequence is from 1-70 amino acids in length. In one aspect, the α-Hemolysin is a α-Hemolysin having at least 95% sequence identity to SEQ ID NO: 3, wherein the α-Hemolysin comprises an amino acid insert sequence between D128 and K131 wherein the amino acid insert sequence is from 1-70 amino acids in length. In one aspect, the α-Hemolysin is a α-Hemolysin having at least 96% sequence identity to SEQ ID NO: 3, wherein the α-Hemolysin comprises an amino acid insert sequence between D128 and K131 wherein the amino acid insert sequence is from 1-70 amino acids in length. In one aspect, the α-Hemolysin is a α-Hemolysin having at least 97% sequence identity to SEQ ID NO: 3, wherein the α-Hemolysin comprises an amino acid insert sequence between D128 and K131 wherein the amino acid insert sequence is from 1-70 amino acids in length. In one aspect, the α-Hemolysin is a α-Hemolysin having at least 98% sequence identity to SEQ ID NO: 3, wherein the α-Hemolysin comprises an amino acid insert sequence between D128 and K131 wherein the amino acid insert sequence is from 1-70 amino acids in length. In one aspect, the α-Hemolysin is a α-Hemolysin having at least 99% sequence identity to SEQ ID NO: 3, wherein the α-Hemolysin comprises an amino acid insert sequence between D128 and K131 wherein the amino acid insert sequence is from 1-70 amino acids in length.

In one aspect, the α-Hemolysin is a α-Hemolysin having at least 80%, 85%, 90%, 91%, 92%, 93%, 94%, 95%, 96%, 97%, 98%, or 99% sequence identity to a 150 amino acid continuous sequence within SEQ ID NO: 3 wherein the α-Hemolysin comprises an amino acid insert sequence between amino acids D128 and K131 wherein the amino acid insert sequence is from 1-70 amino acids in length. In one aspect, the α-Hemolysin is a α-Hemolysin having at least 80%, 85%, 90%, 91%, 92%, 93%, 94%, 95%, 96%, 97%, 98%, or 99% sequence identity to a 150 amino acid continuous sequence within SEQ ID NO: 3 wherein the α-Hemolysin comprises an amino acid insert sequence between amino acids D128 and K131 wherein the amino acid insert sequence is from 1-55 amino acids in length. In one aspect, the α-Hemolysin is a α-Hemolysin having at least 90% sequence identity to a 150 amino acid continuous sequence within SEQ ID NO: 3 wherein the α-Hemolysin comprises an amino acid insert sequence between amino acids D128 and K131 wherein the amino acid insert sequence is from 1-70 amino acids in length. In one aspect, the α-Hemolysin is a α-Hemolysin having at least 98% sequence identity to a 150 amino acid continuous sequence within SEQ ID NO: 3 wherein the α-Hemolysin comprises an amino acid insert sequence between amino acids D128 and K131 wherein the amino acid insert sequence is from 1-70 amino acids in length. In one aspect, the α-Hemolysin is a α-Hemolysin having at least 98% sequence identity to a 150 amino acid continuous sequence within SEQ ID NO: 3 wherein the α-Hemolysin comprises an amino acid insert sequence between amino acids D128 and K131 wherein the amino acid insert sequence is from 1-70 amino acids in length. In one aspect, the α-Hemolysin is a α-Hemolysin having at least 90% sequence identity to a 150 amino acid continuous sequence within SEQ ID NO: 3 wherein the α-Hemolysin comprises an amino acid insert sequence between amino acids D128 and K131 wherein the amino acid insert sequence is from 1-55 amino acids in length. In one aspect, the α-Hemolysin is a α-Hemolysin having at least 98% sequence identity to a 150 amino acid continuous sequence within SEQ ID NO: 3 wherein the α-Hemolysin comprises an amino acid insert sequence between amino acids D128 and K131 wherein the amino acid insert sequence is from 1-55 amino acids in length. In one aspect, the α-Hemolysin is a α-Hemolysin having at least 98% sequence identity to a 150 amino acid continuous sequence within SEQ ID NO: 3 wherein the α-Hemolysin comprises an amino acid insert sequence between amino acids D128 and K131 wherein the amino acid insert sequence is from 1-55 amino acids in length.

In one aspect, the α-Hemolysin is selected from a α-Hemolysin having at least 80%, 85%, 90%, 91%, 92%, 93%, 94%, 95%, 96%, 97%, 98%, or 99% sequence identity to SEQ ID NOs: 4-11. In one aspect, the α-Hemolysin is selected from a α-Hemolysin having at least 90%, sequence identity to SEQ ID NOs: 4-11. In one aspect, the α-Hemolysin is selected from a α-Hemolysin having at least 91%, sequence identity to SEQ ID NOs: 4-11. In one aspect, the α-Hemolysin is selected from a α-Hemolysin having at least 92%, sequence identity to SEQ ID NOs: 4-11. In one aspect, the α-Hemolysin is selected from a α-Hemolysin having at least 93%, sequence identity to SEQ ID NOs: 4-11. In one aspect, the α-Hemolysin is selected from a α-Hemolysin having at least 94%, sequence identity to SEQ ID NOs: 4-11. In one aspect, the α-Hemolysin is selected from a α-Hemolysin having at least 95%, sequence identity to SEQ ID NOs: 4-11. In one aspect, the α-Hemolysin is selected from a α-Hemolysin having at least 96%, sequence identity to SEQ ID NOs: 4-11. In one aspect, the α-Hemolysin is selected from a α-Hemolysin having at least 97%, sequence identity to SEQ ID NOs: 4-11. In one aspect, the α-Hemolysin is selected from a α-Hemolysin having at least 98%, sequence identity to SEQ ID NOs: 4-11.

In an aspect is provided a peptide of the formula AA1-PS1-L1-PS2-AA2 wherein PS1 is an amino acid sequence having at least 90% sequence identity to SEQ ID: 35; PS2 is an amino acid sequence having at least 90% sequence identity to SEQ ID: 36; L1 is a peptidyl linker having 1-70 amino acids; and AA1 and AA2 are independently hydrogen or amino acid sequences having 1-100 amino acids. In embodiments, PS¹ is SEQ ID: 34. In embodiments, PS¹ comprises a Histidine to Leucine mutation corresponding to a position of amino acid H25 of SEQ ID NO:35. In embodiments, L1 is an amino acid insert sequence. In embodiments, L1 is selected from the amino acid insert sequences in Table 2. L1 comprises a cysteine residue. In embodiments, L1 is less than 41 amino acids in length. In embodiments, L1 is less than 52 amino acids in length. In embodiments, L1 comprises a glycine linker.

In embodiments, the amino acid insert sequence comprises a cysteine. In embodiments, the cysteine is at a position corresponding to position 130 in SEQ ID NO: 3. In one aspect, two α-Hemolysins with the amino acid insert sequence comprising a cysteine can form a dimeric α-Hemolysin through the cysteine. In one aspect, the α-Hemolysin is an α-Hemolysin with a H to L mutation at a position corresponding to position 48 in SEQ ID NO:3 (also referred to herein as an “H48L mutation). In one aspect, the α-Hemolysin is a truncated α-Hemolysin. In one aspect, the α-Hemolysin is truncated at the N-terminus. In one aspect, the α-Hemolysin is truncated at the C-terminus. In one aspect, amino acid residues corresponding to amino acids D2-G23 SEQ ID NO:3 are deleted.

TABLE 1
Exemplary alpha α-Hemolysins with and without amino acid insert sequence.

| SEQ ID NO: | Description | SEQUENCE |
|---|---|---|
| 1 | WT- | MKTRIVSSVTTTLLLGSILMNPVAGAADSDINIKTGTTDIGSN |
| | hemolysin | TTVKTGDLVTYDKENGMHKKVFYSFIDDKNHNKKLLVIRT |
| | | KGTIAGQYRVYSEEGANKSGLAWPSAFKVQLQLPDNEVAQI |
| | | SDYYPRNSIDTKEYMSTLTYGFNGNVTGDDTGKIGGLIGAN |
| | | VSIGHTLKYVQPDFKTILESPTDKKVGWKVIFNNMVNQNW |
| | | GPYDRDSWNPVYGNQLFMKTRNGSMKAADNFLDPNKASS |
| | | LLSSGFSPDFATVITMDRKASKQQTNIDVIYERVRDDYQLH |
| | | WTSTNWKGTNTKDKWTDRSSERYKIDWEKEEMTN |
| 3 | Hemolysin | MDSDINIKTGTTDIGSNTTVKTGDLVTYDKENGMHKKVFYS |
| | without | FIDDKNHNKKLLVIRTKGTIAGQYRVYSEEGANKSGLAWPS |
| | Signal | AFKVQLQLPDNEVAQISDYYPRNSIDTKEYMSTLTYGFNGN |
| | Sequence | VTGDDTGKIGGLIGANVSIGHTLKYVQPDFKTILESPTDKKV |
| | | GWKVIFNNMVNQNWGPYDRDSWNPVYGNQLFMKTRNGS |
| | | MKAADNFLDPNKASSLLSSGFSPDFATVITMDRKASKQQTN |
| | | IDVIYERVRDDYQLHWTSTNWKGTNTKDKWTDRSSERYKI |
| | | DWEKEEMTN |
| 4 | hemolysin | MDSDINIKTGTTDIGSNTTVKTGDLVTYDKENGMHKKVFYS |
| | with Cysteine | FIDDKNHNKKLLVIRTKGTIAGQYRVYSEEGANKSGLAWPS |
| | insert | AFKVQLQLPDNEVAQISDYYPRNSIDTKEYMSTLTYGFNGN |
| | | VTGDDGGGGSCGGGGSKIGGLIGANVSIGHTLKYVQPDFKT |
| | | ILESPTDKKVGWKVIFNNMVNQNWGPYDRDSWNPVYGNQ |
| | | LFMKTRNGSMKAADNFLDPNKASSLLSSGFSPDFATVITMD |
| | | RKASKQQTNIDVIYERVRDDYQLHWTSTNWKGTNTKDKW |
| | | TDRSSERYKIDWEKEEMTN |
| 5 | hemolysin | MDSDINIKTGTTDIGSNTTVKTGDLVTYDKENGMHKKVFYS |
| | with XXX | FIDDKNHNKKLLVIRTKGTIAGQYRVYSEEGANKSGLAWPS |
| | insert | AFKVQLQLPDNEVAQISDYYPRNSIDTKEYMSTLTYGFNGN |
| | | VTGDDinsertKIGGLIGANVSIGHTLKYVQPDFKTILESPTDKK |
| | | VGWKVIFNNMVNQNWGPYDRDSWNPVYGNQLFMKTRNG |
| | | SMKAADNFLDPNKASSLLSSGFSPDFATVITMDRKASKQQT |
| | | NIDVIYERVRDDYQLHWTSTNWKGTNTKDKWTDRSSERYK |
| | | IDWEKEEMTN |
| 6 | hemolysin | MDSDINIKTGTTDIGSNTTVKTGDLVTYDKENGMHKKVFYS |
| | with 48H −> | FIDDKNLNKKLLVIRTKGTIAGQYRVYSEEGANKSGLAWPS |
| | L | AFKVQLQLPDNEVAQISDYYPRNSIDTKEYMSTLTYGFNGN |
| | | VTGDDTGKIGGLIGANVSIGHTLKYVQPDFKTILESPTDKKV |
| | | GWKVIFNNMVNQNWGPYDRDSWNPVYGNQLFMKTRNGS |
| | | MKAADNFLDPNKASSLLSSGFSPDFATVITMDRKASKQQTN |
| | | IDVIYERVRDDYQLHWTSTNWKGTNTKDKWTDRSSERYKI |
| | | DWEKEEMTN |
| 7 | hemolysin | MDSDINIKTGTTDIGSNTTVKTGDLVTYDKENGMHKKVFYS |
| | with 48H −> | FIDDKNLNKKLLVIRTKGTIAGQYRVYSEEGANKSGLAWPS |
| | L insert | AFKVQLQLPDNEVAQISDYYPRNSIDTKEYMSTLTYGFNGN |
| | | VTGDDinsertKIGGLIGANVSIGHTLKYVQPDFKTILESPTDKK |
| | | VGWKVIFNNMVNQNWGPYDRDSWNPVYGNQLFMKTRNG |
| | | SMKAADNFLDPNKASSLLSSGFSPDFATVITMDRKASKQQT |
| | | NIDVIYERVRDDYQLHWTSTNWKGTNTKDKWTDRSSERYK |
| | | IDWEKEEMTN |
| 8 | 48H −> L | MDSDINIKTGTTDIGSNTTVKTGDLVTYDKENGMHKKVFYS |
| | Cysteine | FIDDKNLNKKLLVIRTKGTIAGQYRVYSEEGANKSGLAWPS |
| | Insert | AFKVQLQLPDNEVAQISDYYPRNSIDTKEYMSTLTYGFNGN |
| | | VTGDDGGGGSCGGGGSKIGGLIGANVSIGHTLKYVQPDFKT |
| | | ILESPTDKKVGWKVIFNNMVNQNWGPYDRDSWNPVYGNQ |
| | | LFMKTRNGSMKAADNFLDPNKASSLLSSGFSPDFATVITMD |
| | | RKASKQQTNIDVIYERVRDDYQLHWTSTNWKGTNTKDKW |
| | | TDRSSERYKIDWEKEEMTN |
| 9 | hemolysin | MDLVTYDKENGMHKKVFYSFIDDKNHNKKLLVIRTKGTIA |
| | with 2D-23G | GQYRVYSEEGANKSGLAWPSAFKVQLQLPDNEVAQISDYY |
| | truncated | PRNSIDTKEYMSTLTYGFNGNVTGDDTGKIGGLIGANVSIGH |
| | (amino acids | TLKYVQPDFKTILESPTDKKVGWKVIFNNMVNQNWGPYDR |
| | 2D-23G are | DSWNPVYGNQLFMKTRNGSMKAADNFLDPNKASSLLSSGF |
| | missing) | SPDFATVITMDRKASKQQTNIDVIYERVRDDYQLHWTSTNW |
| | | KGTNTKDKWTDRSSERYKIDWEKEEMTN |
| 10 | hemolysin | MDLVTYDKENGMHKKVFYSFIDDKNHNKKLLVIRTKGTIA |
| | with 2D-23G | GQYRVYSEEGANKSGLAWPSAFKVQLQLPDNEVAQISDYY |
| | truncated | PRNSIDTKEYMSTLTYGFNGNVTGDDinsertKIGGLIGANVSI |
| | (amino acids | GHTLKYVQPDFKTILESPTDKKVGWKVIFNNMVNQNWGPY |
| | 2D-23G are | DRDSWNPVYGNQLFMKTRNGSMKAADNFLDPNKASSLLSS |
| | missing) with | GFSPDFATVITMDRKASKQQTNIDVIYERVRDDYQLHWTST |
| | insert | NWKGTNTKDKWTDRSSERYKIDWEKEEMTN |
| 11 | hemolysin | MDLVTYDKENGMHKKVFYSFIDDKNHNKKLLVIRTKGTIA |
| | with 2D-23G | GQYRVYSEEGANKSGLAWPSAFKVQLQLPDNEVAQISDYY |
| | truncated | PRNSIDTKEYMSTLTYGFNGNVTGDDGGGGSCGGGGSKIGG |
| | (amino acids | LIGANVSIGHTLKYVQPDFKTILESPTDKKVGWKVIFNNMV |
| | 2D-23G are | NQNWGPYDRDSWNPVYGNQLFMKTRNGSMKAADNFLDP |
| | missing) with | NKASSLLSSGFSPDFATVITMDRKASKQQTNIDVIYERVRDD |
| | insert | YQLHWTSTNWKGTNTKDKWTDRSSERYKIDWEKEEMTN |

In an aspect is provided an α-Hemolysin with a modification in the loop (e.g., corresponding to amino acids 154-157 (SEQ ID NO: 1) or corresponding to amino acids 128-131 (SEQ ID NO:3)). In one aspect the modification is a deletion of amino acids. In one aspect the modification is an insertion of amino acids. In one aspect the modification is a combined deletion and an insertion of amino acids. In an aspect, the deletion comprises amino acids corresponding to position 154-157 (SEQ ID NO: 1) or 128-131 (SEQ ID NO:3). In one aspect amino acids 129-130 in SEQ ID NO:3 are deleted. In one aspect the insertion is between amino acids 154-157 (SEQ ID NO: 1) or 128-131 (SEQ ID NO:3). In one aspect the insertion is between residues corresponding to amino acids 128-131 (SEQ ID NO:3) and amino acids 129-130 in SEQ ID NO:3 are deleted.

In one aspect, the amino acid insert sequence (also referred to herein as an “insertion”) is greater than 2 amino acids. In one aspect, the insertion is greater than 3 amino acids. In one aspect, the insertion is greater than 4 amino acids. In one aspect, the insertion is greater than 5 amino acids. In one aspect, the insertion is greater than 6 amino acids. In one aspect, the insertion is greater than 7 amino acids. In one aspect, the insertion is greater than 8 amino acids. In one aspect, the insertion is greater than 9 amino acids. In one aspect, the insertion is greater than 10 amino acids. In one aspect, the insertion is greater than 11 amino acids. In one aspect, the insertion is greater than 12 amino acids. In one aspect, the insertion is greater than 13 amino acids. In one aspect, the insertion is greater than 14 amino acids. In one aspect, the insertion is greater than 15 amino acids. In one aspect, the insertion is greater than 16 amino acids. In one aspect, the insertion is greater than 17 amino acids. In one aspect, the insertion is greater than 18 amino acids. In one aspect, the insertion is greater than 19 amino acids. In one aspect, the insertion is greater than 20 amino acids. In one aspect, the insertion is greater than 25 amino acids. In one aspect, the insertion is greater than 30 amino acids. In one aspect, the insertion is greater than 35 amino acids. In one aspect, the insertion is greater than 40 amino acids. In one aspect, the insertion is greater than 45 amino acids. In one aspect, the insertion is greater than 50 amino acids. In one aspect, the insertion is greater than 55 amino acids. In one aspect, the insertion is greater than 60 amino acids. In one aspect, the insertion is greater than 65 amino acids. In one aspect, the insertion is greater than 70 amino acids. In one aspect, the insertion is greater than 75 amino acids. In one aspect, the insertion is greater than 80 amino acids. In one aspect, the insertion is greater than 85 amino acids. In one aspect, the insertion is greater than 90 amino acids. In one aspect, the insertion is greater than 95 amino acids. In one aspect, the insertion is greater than 100 amino acids.

In one aspect, the insertion is greater than 5 amino acids and less than 41 amino acids. In one aspect, the insertion is greater than 5 amino acids and less than 53 amino acids.

In one aspect the amino acid insert sequence is a protein domain. In one aspect, the amino acid insert sequence comprises a linker. In one aspect the amino acid insert sequence comprises a protein domain and one or more linkers. In one aspect the amino acid insert sequence has the structure Linker-protein domain-Linker. In one aspect the amino acid insert sequence has the structure Linker1-protein domain-Linker2.

In one aspect, the amino acid insert sequence comprises a cyclic peptide. In one aspect, the amino acid insert sequence comprises a Somatostatin sequence. In one aspect, the amino acid insert sequence comprises a Somatostatin 14 sequence. In one aspect, the amino acid insert sequence comprises a GLP1 sequence. In one aspect, the insertion comprises cell adhesion molecules. In one aspect, the amino acid insert sequence comprises a K3 Sequence. In one aspect, the amino acid insert sequence comprises an E3 sequence. In one aspect, the amino acid insert sequence comprises a Sac7e Sequence. In one aspect, the amino acid insert sequence comprises a SNAP sequence. In one aspect, the amino acid insert sequence comprises an amino acid sequence selected from Table 2. In one aspect, the amino acid insert sequence comprises an amino acid sequence selected from SEQ ID NO: 21 to SEQ ID NO: 32.

TABLE 2
Exemplary amino acid insert sequences

| SEQ ID NO: | Description | SEQUENCE |
|---|---|---|
| 21 | Linker 1 (L1) | GGGGS |
| 22 | Linker 2 (L2) | GGGGSGGGGS |
| 23 | Linker 3 (L3) | GGGGSGGGGSGGGGS |
| 24 | L1-6XHis-L1 | GGGGSHHHHHHGGGGS |
| 25 | L2-6XHis-L2 | GGGGSGGGGSHHHHHHGGGGSGGGGS |
| 26 | L3-6XHis-L3 | GGGGSGGGGSGGGGSHHHHHHGGGGSGGGGSGGGGS |
| 27 | L1-Somatostatin-L1 | GGGGSAGCKNFFWKTFTSCGGGGS |
| 28 | L2-GLP1-L2 | GGGGSGGGGSHAEGTFTSDVSSYLEGQAAKEFIAWLVKG |
| | | RGGGGSGGGGS |
| 29 | L1-K3-L1 | GGGGSKGGKGGKGGGGS |
| 30 | L1-E3-L1 | GGGGSEGGEGGEGGGGS |
| 31 | L2-Sac7e-L2 | GGGGSGGGGSMAKVRFKYKGEEKEVDTSKIKKVWRVG |
| | | KMVSFTYDDNGKTGRGAVSEKDAPKELMDMLARAEKK |
| | | KGGGGSGGGGS |
| 32 | L2-SNAP-L2 | GGGGSGGGGSMDKDCEMKRTTLDSPLGKLELSGCEQGL |
| | | HRIIFLGKGTSAADAVEVPAPAAVLGGPEPLMQATAWLN |
| | | AYFHQPEAIEEFPVPALHHPVFQQESFTRQVLWKLLKVVK |
| | | FGEVISYSHLAALAGNPAATAAVKTALSGNPVPILIPCHR |
| | | VVQGDLDVGGYEGGLAVKEWLLAHEGHRLGKPGLGGG |
| | | GGSGGGGS |

In one aspect, the α-Hemolysin is selected from a α-Hemolysin listed in Table 3. In one aspect, the α-Hemolysin is selected from SEQ ID NOs: 12-20. In one aspect, the α-Hemolysin is an α-Hemolysin having at least 80%, 85%, 90%, 91%, 92%, 93%, 94%, 95%, 96%, 97%, 98%, or 99% sequence identity to SEQ ID NOs: 12-20. In one aspect, the α-Hemolysin is a α-Hemolysin having at least 80% sequence identity to SEQ ID NOs: 12-20. In one aspect, the α-Hemolysin is an α-Hemolysin having at least 85% sequence identity to a sequence selected from SEQ ID NOs: 12-20. In one aspect, the α-Hemolysin is a α-Hemolysin having at least 90% sequence identity to a sequence selected from SEQ ID NOs: 12-20. In one aspect, the α-Hemolysin is a α-Hemolysin having at least 91% sequence identity to a sequence selected from SEQ ID NOs: 12-20. In one aspect, the α-Hemolysin is a α-Hemolysin having at least 92% sequence identity to a sequence selected from SEQ ID NOs: 12-20. In one aspect, the α-Hemolysin is a α-Hemolysin having at least 93% sequence identity to a sequence selected from SEQ ID NOs: 12-20. In one aspect, the α-Hemolysin is a α-Hemolysin having at least 94% sequence identity to a sequence selected from SEQ ID NOs: 12-20. In one aspect, the α-Hemolysin is a α-Hemolysin having at least 95% sequence identity to sequence selected from SEQ ID NOs: 12-20. In one aspect, the α-Hemolysin is a α-Hemolysin having at least 96% sequence identity to a sequence selected from SEQ ID NOs: 12-20. In one aspect, the α-Hemolysin is a α-Hemolysin having at least 97% sequence identity to a sequence selected from SEQ ID NOs: 12-20. In one aspect, the α-Hemolysin is a α-Hemolysin having at least 98% sequence identity to a sequence selected from SEQ ID NOs: 12-20. In one aspect, the α-Hemolysin is a α-Hemolysin having at least 99% sequence identity to a sequence selected from SEQ ID NOs: 12-20. In embodiments, the α-Hemolysin does not contain the N-terminal methionine set forth in the α-Hemolysin sequences of Table 3.

TABLE 3
Exemplary α-Hemolysins.

| SEQ ID NO: | Description | SEQUENCE |
|---|---|---|
| 12 | hemolysin | MDSDINIKTGTTDIGSNTTVKTGDLVTYDKENGMHKKVFYSFIDD |
| | with L- | KNHNKKLLVIRTKGTIAGQYRVYSEEGANKSGLAWPSAFKVQLQ |
| | 6XHis-L | LPDNEVAQISDYYPRNSIDTKEYMSTLTYGFNGNVTGDDGGGGSH |
| | | HHHHHGGGGSKIGGLIGANVSIGHTLKYVQPDFKTILESPTDKKV |
| | | GWKVIFNNMVNQNWGPYDRDSWNPVYGNQLFMKTRNGSMKAA |
| | | DNFLDPNKASSLLSSGFSPDFATVITMDRKASKQQTNIDVIYERVR |
| | | DDYQLHWTSTNWKGTNTKDKWTDRSSERYKIDWEKEEMTN |
| 13 | hemolysin | MDSDINIKTGTTDIGSNTTVKTGDLVTYDKENGMHKKVFYSFIDD |
| | with L2- | KNHNKKLLVIRTKGTIAGQYRVYSEEGANKSGLAWPSAFKVQLQ |
| | 6XHis-L2 | LPDNEVAQISDYYPRNSIDTKEYMSTLTYGFNGNVTGDDGGGGSG |
| | | GGGSHHHHHHGGGGSGGGGSKIGGLIGANVSIGHTLKYVQPDFK |
| | | TILESPTDKKVGWKVIFNNMVNQNWGPYDRDSWNPVYGNQLFM |
| | | KTRNGSMKAADNFLDPNKASSLLSSGFSPDFATVITMDRKASKQQ |
| | | TNIDVIYERVRDDYQLHWTSTNWKGTNTKDKWTDRSSERYKIDW |
| | | EKEEMTN |
| 14 | hemolysin | MDSDINIKTGTTDIGSNTTVKTGDLVTYDKENGMHKKVFYSFIDD |
| | with L3- | KNHNKKLLVIRTKGTIAGQYRVYSEEGANKSGLAWPSAFKVQLQ |
| | 6XHis-L3 | LPDNEVAQISDYYPRNSIDTKEYMSTLTYGFNGNVTGDDGGGGSG |
| | | GGGSGGGGSHHHHHHGGGGSGGGGSGGGGSKIGGLIGANVSIGH |
| | | TLKYVQPDFKTILESPTDKKVGWKVIFNNMVNQNWGPYDRDSW |
| | | NPVYGNQLFMKTRNGSMKAADNFLDPNKASSLLSSGFSPDFATVI |
| | | TMDRKASKQQTNIDVIYERVRDDYQLHWTSTNWKGTNTKDKWT |
| | | DRSSERYKIDWEKEEMTN |
| 15 | hemolysin | MDSDINIKTGTTDIGSNTTVKTGDLVTYDKENGMHKKVFYSFIDD |
| | with L- | KNHNKKLLVIRTKGTIAGQYRVYSEEGANKSGLAWPSAFKVQLQ |
| | Somatosta | LPDNEVAQISDYYPRNSIDTKEYMSTLTYGFNGNVTGDDGGGGSA |
| | tin-L | GCKNFFWKTFTSCGGGGSKIGGLIGANVSIGHTLKYVQPDFKTILE |
| | | SPTDKKVGWKVIFNNMVNQNWGPYDRDSWNPVYGNQLFMKTR |
| | | NGSMKAADNFLDPNKASSLLSSGFSPDFATVITMDRKASKQQTNI |
| | | DVIYERVRDDYQLHWTSTNWKGTNTKDKWTDRSSERYKIDWEK |
| | | EEMTN |
| 16 | hemolysin | MDSDINIKTGTTDIGSNTTVKTGDLVTYDKENGMHKKVFYSFIDD |
| | with L2- | KNHNKKLLVIRTKGTIAGQYRVYSEEGANKSGLAWPSAFKVQLQ |
| | GLP1-L2 | LPDNEVAQISDYYPRNSIDTKEYMSTLTYGFNGNVTGDDGGGGSG |
| | | GGGSHAEGTFTSDVSSYLEGQAAKEFIAWLVKGRGGGGSGGGGS |
| | | KIGGLIGANVSIGHTLKYVQPDFKTILESPTDKKVGWKVIFNNMV |
| | | NQNWGPYDRDSWNPVYGNQLFMKTRNGSMKAADNFLDPNKAS |
| | | SLLSSGFSPDFATVITMDRKASKQQTNIDVIYERVRDDYQLHWTST |
| | | NWKGTNTKDKWTDRSSERYKIDWEKEEMTN |
| 17 | hemolysin | MDSDINIKTGTTDIGSNTTVKTGDLVTYDKENGMHKKVFYSFIDD |
| | with L- | KNHNKKLLVIRTKGTIAGQYRVYSEEGANKSGLAWPSAFKVQLQ |
| | K3-L | LPDNEVAQISDYYPRNSIDTKEYMSTLTYGFNGNVTGDDGGGGSK |
| | | GGKGGKGGGGSKIGGLIGANVSIGHTLKYVQPDFKTILESPTDKK |
| | | VGWKVIFNNMVNQNWGPYDRDSWNPVYGNQLFMKTRNGSMKA |
| | | ADNFLDPNKASSLLSSGFSPDFATVITMDRKASKQQTNIDVIYERV |
| | | RDDYQLHWTSTNWKGTNTKDKWTDRSSERYKIDWEKEEMTN |
| 18 | hemolysin | MDSDINIKTGTTDIGSNTTVKTGDLVTYDKENGMHKKVFYSFIDD |
| | with L- | KNHNKKLLVIRTKGTIAGQYRVYSEEGANKSGLAWPSAFKVQLQ |
| | E3-L | LPDNEVAQISDYYPRNSIDTKEYMSTLTYGFNGNVTGDDGGGGSE |
| | | GGEGGEGGGGSKIGGLIGANVSIGHTLKYVQPDFKTILESPTDKKV |
| | | GWKVIFNNMVNQNWGPYDRDSWNPVYGNQLFMKTRNGSMKAA |
| | | DNFLDPNKASSLLSSGFSPDFATVITMDRKASKQQTNIDVIYERVR |
| | | DDYQLHWTSTNWKGTNTKDKWTDRSSERYKIDWEKEEMTN |
| 19 | hemolysin | MDSDINIKTGTTDIGSNTTVKTGDLVTYDKENGMHKKVFYSFIDD |
| | with L2- | KNHNKKLLVIRTKGTIAGQYRVYSEEGANKSGLAWPSAFKVQLQ |
| | Sac7e-L2 | LPDNEVAQISDYYPRNSIDTKEYMSTLTYGFNGNVTGDDGGGGSG |
| | | GGGSMAKVRFKYKGEEKEVDTSKIKKVWRVGKMVSFTYDDNGK |
| | | TGRGAVSEKDAPKELMDMLARAEKKKGGGGSGGGGSKIGGLIGA |
| | | NVSIGHTLKYVQPDFKTILESPTDKKVGWKVIFNNMVNQNWGPY |
| | | DRDSWNPVYGNQLFMKTRNGSMKAADNFLDPNKASSLLSSGFSP |
| | | DFATVITMDRKASKQQTNIDVIYERVRDDYQLHWTSTNWKGTNT |
| | | KDKWTDRSSERYKIDWEKEEMTN |
| 20 | hemolysin | MDSDINIKTGTTDIGSNTTVKTGDLVTYDKENGMHKKVFYSFIDD |
| | with L2- | KNHNKKLLVIRTKGTIAGQYRVYSEEGANKSGLAWPSAFKVQLQ |
| | SNAP-L2 | LPDNEVAQISDYYPRNSIDTKEYMSTLTYGFNGNVTGDDGGGGSG |
| | | GGGSMDKDCEMKRTTLDSPLGKLELSGCEQGLHRIIFLGKGTSAA |
| | | DAVEVPAPAAVLGGPEPLMQATAWLNAYFHQPEAIEEFPVPALH |
| | | HPVFQQESFTRQVLWKLLKVVKFGEVISYSHLAALAGNPAATAA |
| | | VKTALSGNPVPILIPCHRVVQGDLDVGGYEGGLAVKEWLLAHEG |
| | | HRLGKPGLGGGGGSGGGGSKIGGLIGANVSIGHTLKYVQPDFKTI |
| | | LESPTDKKVGWKVIFNNMVNQNWGPYDRDSWNPVYGNQLFMK |
| | | TRNGSMKAADNFLDPNKASSLLSSGFSPDFATVITMDRKASKQQT |
| | | NIDVIYERVRDDYQLHWTSTNWKGTNTKDKWTDRSSERYKIDWE |
| | | KEEMTN |

In one aspect, the α-Hemolysin comprises an N or C terminal fusion protein. In embodiments, the fusion protein is AA¹ and/or AA². In embodiments, the fusion protein is a tag. In one aspect, the fusion protein is a signal sequence. In one aspect, the fusion protein is a protein tag. In one aspect, the fusion protein is a fluorescent protein. In one aspect, the fluorescent protein is a GFP protein. In one aspect, the fluorescent protein is a GFP, an eGFP, a RFP, a YFP, a BFP, or a CFP. In one aspect, the α-Hemolysin comprises a targeting protein or peptide. In one aspect, the targeting protein or peptide targets the α-Hemolysin to a specific cell type. In one aspect, the targeting protein is an antibody or an antibody fragment. In one aspect, the targeting protein is galectin-1 (Gal1) (see e.g., Bayley et al. ACS Cent. Sci. (2019) 5, 629-629).

In an aspect, the pore forming toxin (e.g., alpha α-Hemolysin) is soluble.

The monomeric α-hemolysin self assembles into a multimer (e.g., a heptamer). In one aspect, an α-Hemolysin multimer comprising two or more α-hemolysin monomers is provided. In one aspect, the α-Hemolysin multimer is a heptamer. In one aspect, the α-Hemolysin heptamer is formed by seven α-hemolysin monomers. In one aspect, the α-hemolysin monomers are the same. In one aspect, the α-hemolysin monomers are different. In one aspect, six α-hemolysin monomers comprise a cysteine insertion in the amino acid insert sequence. In one aspect, six of the seven α-hemolysin monomers in a α-Hemolysin heptamer comprise a cysteine insertion in the amino acid insert sequence and one α-hemolysin monomer comprises no cysteine insertion in the amino acid insert sequence. In one aspect, six of the seven α-hemolysin monomers of an α-Hemolysin heptamer comprise a cysteine mutation in a position corresponding to G130 of SEQ ID NO:3 and one monomer comprises no cysteine insertion in the amino acid insert sequence. In one aspect, the amino acid insert sequence is the same in all seven α-hemolysin monomers in a α-Hemolysin heptamer. In one aspect, the amino acid insert sequence is different in all seven α-hemolysin monomers in a α-Hemolysin heptamer.

In one aspect, a mixture of α-hemolysin monomers is provided. In one aspect, the mixture comprises α-hemolysin monomers with a cysteine mutation in a position corresponding to G130 of SEQ ID NO:3 (hereinafter “G130C α-hemolysin monomer”) and α-hemolysin monomers without a cysteine in the amino acid insert sequence. In one aspect, the ratio of G130C α-hemolysin monomers and α-hemolysin monomers without a cysteine insertion in the amino acid insert sequence 1:1. In one aspect, the ratio of G130C α-hemolysin monomers and α-hemolysin monomers without a cysteine insertion in the amino acid insert sequence is 2:1. In one aspect, the ratio of G130C α-hemolysin monomers and α-hemolysin monomers without a cysteine insertion in the intracellular loop is 3:1. In one aspect, the ratio of G130C α-hemolysin monomers and α-hemolysin monomers without a cysteine insertion in the amino acid insert sequence 4:1. In one aspect, the ratio of G130C α-hemolysin monomers and α-hemolysin monomers without a cysteine insertion in the amino acid insert sequence is 5:1. In one aspect, the ratio of G130C α-hemolysin monomers and α-hemolysin monomers without a cysteine insertion in the amino acid insert sequence is 6:1.

The terms “polypeptide,” “peptide” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

Provided herein are, inter alia, nucleic acids encoding the pore forming toxins and α-Hemolysins and the α-Hemolysins described herein.

In some embodiments, the nucleic acid is a DNA or RNA In some embodiments, the DNA is circular plasmid DNA, linear double-strand DNA, single strand DNA, or chimeric RNA and DNA.

In some embodiments, the RNA is mRNA. In some embodiments, the mRNA comprises nucleic acid mimetics selected from the group of peptide nucleic acid (PNA), morpholino nucleic acid, cyclohexenyl nucleic acid (CeNAs), and locked nucleic acid (LNA). In some embodiments, the mRNA comprises modified sugar moieties, optionally wherein the modified sugar moiety is selected from the group of N1-methylpseudouridine, 9-Methyladenine, 2′-O-(2-methoxyethyl), 2′-dimethylaminooxyethoxy, 2′-dimethylaminoethoxyethoxy, 2′-O-methyl, and 2′-fluoro. In some embodiments, the mRNA comprises a modified nucleobase, optionally wherein the modified nucleobase is selected from the group of a 5-methylcytosine; a 5-hydroxymethyl cytosine; a xanthine; a hypoxanthine; a 2-aminoadenine; a 6-methyl derivative of adenine; a 6-methyl derivative of guanine; a 2-propyl derivative of adenine; a 2-propyl derivative of guanine; a 2-thiouracil; a 2-thiothymine; a 2-thiocytosine; a 5-halouracil; a 5-halocytosine; a 5-propynyl uracil; a 5-propynyl cytosine; a 6-azo uracil; a 6-azo cytosine; a 6-azo thymine; a pseudouracil; a 4-thiouracil; an 8-halo; an 8-amino; an 8-thiol; an 8-thioalkyl; an 8-hydroxyl; a 5-halo; a 5-bromo; a 5-trifluoromethyl; a 5-substituted uracil; a 5-substituted cytosine; a 7-methylguanine; a 7-methyladenine; a 2-F-adenine; a 2-amino-adenine; an 8-azaguanine; an 8-azaadenine; a 7-deazaguanine; a 7-deazaadenine; a 3-deazaguanine; a 3-deazaadenine; a tricyclic pyrimidine; a phenoxazine cytidine; a phenothiazine cytidine; a substituted phenoxazine cytidine; a carbazole cytidine; a pyridoindole cytidine; a 7-deaza-adenine; a 7-deazaguanosine; a 2-aminopyridine; a 2-pyridone; a 5-substituted pyrimidine; a 6-azapyrimidine; an N-2, N-6 or 0-6 substituted purine; a 2-aminopropyladenine; a 5-propynyluracil; or a 5-propynylcytosine. In some embodiments, the mRNA comprises a non-naturally occurring or a non-natural internucleoside linkage selected from the group of a phosphorothioate, a phosphoramidate, a non-phosphodiester, a heteroatom, a chiral phosphorothioate, a phosphorodithioate, a phosphotriester, an aminoalkylphosphotriester, a 3′-alkylene phosphonates, a 5′-alkylene phosphonate, a chiral phosphonate, a phosphinate, a 3′-amino phosphoramidate, an aminoalkylphosphoramidate, a phosphorodiamidate, a thionophosphoramidate, a thionoalkylphosphonate, a thionoalkylphosphotriester, a selenophosphate, or a boranophosphate.

### III. Bioconjugates

Provided herein are pore forming toxin of the disclosure (e.g., a beta barrel pore forming toxin) bound to a bioconjugate.

In an aspect is provided a α-Hemolysin bound to a bioconjugate, the α-Hemolysin comprising an amino acid insert sequence between the residues corresponding to residues D154 and K157 of SEQ ID NO: 1 or 128-131 (SEQ ID NO:3), wherein the amino acid insert sequence is from 1-70 amino acids in length.

In an aspect is provided a peptide bound to a bioconjugate, wherein the peptide has the formula: AA¹-PS¹-L¹-PS²-AA² wherein PS¹ is an amino acid sequence having at least 90% sequence identity to SEQ ID: 35; PS² is an amino acid sequence having at least 90% sequence identity to SEQ ID: 36; L¹ is a peptidyl linker having 1-70 amino acids; and AA¹ and AA² are independently hydrogen or amino acid sequences having 1-100 amino acids.

In one aspect, the bioconjugate is bound to the polypeptide chain of the pore forming toxin. In one aspect, the bioconjugate is bound to the polypeptide chain of the α-Hemolysin. In one aspect, the bioconjugate is bound to the polypeptide chain of the AA¹-PS¹-L¹-PS²-AA² peptide. In one aspect, the bioconjugate is bound to the amino acid insert sequence. In one aspect, the bioconjugate is bound to an amino acid residue in L¹. In one aspect, the bioconjugate is bound to an amino acid residue of the amino acid insert sequence (e.g., within or replacing amino acids 154-157 (SEQ ID NO: 1) or 128-131 (SEQ ID NO:3). In one aspect, the bioconjugate is bound to a cysteine residue in the amino acid insert sequence.

In one aspect, the bioconjugate is bound to a cargo molecule. In one aspect, the bioconjugate binds the cargo molecule to the pore forming toxin. In one aspect, the cargo is a polynucleotide. In embodiments, the cargo molecule is a biomolecule. In embodiments, the cargo molecule is a small molecule. In embodiments, the cargo molecule is a drug. In one aspect, the polynucleotide is a DNA RNA, siRNA, mRNA, miRNA, aptamer, or antisense oligonucleotide. In one aspect, the DNA is a plasmid DNA. In one aspect, the polynucleotide encodes a peptide or a polypeptide. In one aspect, the polynucleotide encodes a therapeutic peptide or a polypeptide. In one aspect, the peptide is a cyclic peptide. In one aspect, the cargo is a peptide or a polypeptide. In one aspect, the cargo is an enzyme. In one aspect, the cargo is a therapeutic peptide or protein. In one aspect, the cargo is a cell adhesion molecule. In one aspect, the polypeptide is an antibody or an antibody fragment. In one aspect, the cargo is a gene editing system. In one aspect, the cargo is a CRISPR nuclease. In one aspect, the cargo is a small molecule. In one aspect, the cargo is a lipid. In one aspect, the cargo is a proteolysis targeting chimera (PROTAC). In one aspect, the cargo is a nanoparticle. In one aspect, the cargo is a polymer. In one aspect, the cargo is a carbohydrate.

In some embodiments, the DNA is circular plasmid DNA, linear double-strand DNA, single strand DNA, or chimeric RNA and DNA.

In some embodiments, the RNA is mRNA. In some embodiments, the mRNA comprises nucleic acid mimetics selected from the group of peptide nucleic acid (PNA), morpholino nucleic acid, cyclohexenyl nucleic acid (CeNAs), and locked nucleic acid (LNA). In some embodiments, the mRNA comprises modified sugar moieties, optionally wherein the modified sugar moiety is selected from the group of N1-methylpseudouridine, 9-Methyladenine, 2′-O-(2-methoxyethyl), 2′-dimethylaminooxyethoxy, 2′-dimethylaminoethoxyethoxy, 2′-O-methyl, and 2′-fluoro. In some embodiments, the mRNA comprises a modified nucleobase, optionally wherein the modified nucleobase is selected from the group of a 5-methylcytosine; a 5-hydroxymethyl cytosine; a xanthine; a hypoxanthine; a 2-aminoadenine; a 6-methyl derivative of adenine; a 6-methyl derivative of guanine; a 2-propyl derivative of adenine; a 2-propyl derivative of guanine; a 2-thiouracil; a 2-thiothymine; a 2-thiocytosine; a 5-halouracil; a 5-halocytosine; a 5-propynyl uracil; a 5-propynyl cytosine; a 6-azo uracil; a 6-azo cytosine; a 6-azo thymine; a pseudouracil; a 4-thiouracil; an 8-halo; an 8-amino; an 8-thiol; an 8-thioalkyl; an 8-hydroxyl; a 5-halo; a 5-bromo; a 5-trifluoromethyl; a 5-substituted uracil; a 5-substituted cytosine; a 7-methylguanine; a 7-methyladenine; a 2-F-adenine; a 2-amino-adenine; an 8-azaguanine; an 8-azaadenine; a 7-deazaguanine; a 7-deazaadenine; a 3-deazaguanine; a 3-deazaadenine; a tricyclic pyrimidine; a phenoxazine cytidine; a phenothiazine cytidine; a substituted phenoxazine cytidine; a carbazole cytidine; a pyridoindole cytidine; a 7-deaza-adenine; a 7-deazaguanosine; a 2-aminopyridine; a 2-pyridone; a 5-substituted pyrimidine; a 6-azapyrimidine; an N-2, N-6 or O-6 substituted purine; a 2-aminopropyladenine; a 5-propynyluracil; or a 5-propynylcytosine. In some embodiments, the mRNA comprises a non-naturally occurring or a non-natural internucleoside linkage selected from the group of a phosphorothioate, a phosphoramidate, a non-phosphodiester, a heteroatom, a chiral phosphorothioate, a phosphorodithioate, a phosphotriester, an aminoalkylphosphotriester, a 3′-alkylene phosphonates, a 5′-alkylene phosphonate, a chiral phosphonate, a phosphinate, a 3′-amino phosphoramidate, an aminoalkylphosphoramidate, a phosphorodiamidate, a thionophosphoramidate, a thionoalkylphosphonate, a thionoalkylphosphotriester, a selenophosphate, or a boranophosphate.

As used herein, the terms “bioconjugate” and “bioconjugate linker” refers to the resulting association between atoms or molecules of “bioconjugate reactive groups” or “bioconjugate reactive moieties”. The association can be direct or indirect. For example, a conjugate between a first bioconjugate reactive group (e.g., —NH₂, —C(O)OH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

In one aspect, the first bioconjugate reactive group is attached to the pore forming toxin. In one aspect, the second bioconjugate reactive group is attached to the cargo molecule.

In one aspect, the first bioconjugate reactive group is attached to the cargo molecule. In one aspect, the second bioconjugate reactive group is attached to the pore forming toxin.

In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

In embodiments, bioconjugate chemistries include carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters. In embodiments, bioconjugate chemistries include hydroxyl groups which can be converted to esters, ethers, aldehydes, etc. In embodiments, bioconjugate chemistries include haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom. In embodiments, bioconjugate chemistries include dienophile groups which are capable of participating in Diels-Alder reactions such as, for example, maleimido or maleimide groups. In embodiments, bioconjugate chemistries include aldehyde or ketone groups such that subsequent derivatization is possible via formation of carbonyl derivatives such as, for example, imines, hydrazones, semicarbazones or oximes, or via such mechanisms as Grignard addition or alkyllithium addition. In embodiments, bioconjugate chemistries include sulfonyl halide groups for subsequent reaction with amines, for example, to form sulfonamides. In embodiments, bioconjugate chemistries include thiol groups, which can be converted to disulfides, reacted with acyl halides, or bonded to metals such as gold, or react with maleimides.

In embodiments, bioconjugate chemistries include amine or sulfhydryl groups (e.g., present in cysteine), which can be, for example, acylated, alkylated or oxidized. In embodiments, bioconjugate chemistries include alkenes, which can undergo, for example, cycloadditions, acylation, Michael addition, etc. In embodiments, bioconjugate chemistries include epoxides, which can react with, for example, amines and hydroxyl compounds. In embodiments, bioconjugate chemistries include phosphoramidites and other standard functional groups useful in nucleic acid synthesis. In embodiments, bioconjugate chemistries include metal silicon oxide bonding. In embodiments, bioconjugate chemistries include metal bonding to reactive phosphorus groups (e.g., phosphines) to form, for example, phosphate diester bonds. In embodiments, bioconjugate chemistries include azides coupled to alkynes using copper catalyzed cycloaddition click chemistry. In embodiments, bioconjugate chemistries include biotin conjugate can react with avidin or streptavidin to form an avidin-biotin complex or streptavidin-biotin complex.

In one aspect, the bioconjugate is cleavable. In one aspect, the bioconjugate is cleavable by a protease. In one aspect, the bioconjugate includes a disulfide bond between the α-Hemolysin and the cargo. In one aspect, the bioconjugate releases the cargo after cleavage. In one aspect, the bioconjugate disulfide bond is cleavable.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

In one aspect, α-Hemolysin multimer (e.g., a heptamer) bound to one or more bioconjugates is provided. In one aspect, the α-Hemolysin multimer is a heptamer. In one aspect, the each of the seven α-hemolysin monomers are bound to a bioconjugate. In one aspect, the six of the seven α-hemolysin monomers are bound to a bioconjugate. In one aspect, the five of the seven α-hemolysin monomers are bound to a bioconjugate. In one aspect, the four of the seven α-hemolysin monomers are bound to a bioconjugate. In one aspect, the three of the seven α-hemolysin monomers are bound to a bioconjugate. In one aspect, the two of the seven α-hemolysin monomers are bound to a bioconjugate. In one aspect, the one of the seven α-hemolysin monomers is bound to a bioconjugate. In one aspect, the bioconjugates are different. In one aspect, the bioconjugates are the same. In one aspect, the α-hemolysin monomers are different. In one aspect, the α-hemolysin monomers are the same.

In embodiments, the cargo molecule may be delivered to the intracellular space of a cell or lipid bilayer vesicle using the methods described herein.

### IV. Cells and Lipid Vesicles

Provided herein are cells comprising the pore forming toxins (e.g., a beta barrel pore forming toxin) of the disclosure.

Provided herein are lipid vesicles (e.g., a phospholipid membrane) comprising the pore forming toxins (e.g., a beta barrel pore forming toxin) of the disclosure.

In an aspect, the cell is a eukaryotic cell. In some embodiments, the cell is a mammalian cell. In some embodiments, the cell is a human cell. In some embodiments, the cell is an immune cells (such as T-cells), hematopoietic stems cells, mesenchymal stem cells, or induced pluripotent stem cells (iPSCs). In some embodiments, the cell is a cancer cell.

In an aspect, the cell is an artificial cell. In an aspect, the lipid vesicles membrane composition is 1,2-Dioleoyl-sn-glycero-3-phosphocholine (DOPC) (60%) and cholesterol (40%). In an aspect, the lipid vesicles membrane composition comprises DOPC and cholesterol. In an aspect, the lipid vesicles membrane composition is phosphatidylcholine (POPC) (60%) and cholesterol (40%). In an aspect, the lipid vesicles membrane composition comprises POPC and cholesterol.

In an aspect, the lipid vesicle (e.g., phospholipid membrane) additionally comprises a macromolecule. In an aspect, the macromolecule is a polypeptide or polynucleotide. In an aspect, the polynucleotide is DNA or RNA. In an aspect, the polynucleotide is mRNA, siRNA, miRNA, or microRNA. In an aspect, the macromolecule comprises a bioconjugate reactive moiety. In an aspect, the bioconjugate reactive moiety is a maleimide. In an aspect, the macromolecule comprises a bioconjugate.

In an aspect, the pore of the pore forming toxin is facing outside-in in the lipid membrane. In an aspect, the pore of the pore forming toxin is facing inside-out in the lipid membrane.

In an aspect, a tissue-like structure comprising a plurality of phospholipid membranes comprising the pore forming toxins of the disclosure is provided.

### V. Methods of Use

Provided herein are methods of using the pore forming toxins (e.g., a beta barrel pore forming toxin) of the disclosure.

In an aspect, a method of delivering a cargo to a cell is provided. In an aspect, the method comprises contacting the cell with a α-Hemolysin of the disclosure attached to a cargo.

In an aspect, a method of delivering a cargo to a lipid vesicle is provided. In an aspect, the method comprises contacting the lipid vesicle with a α-Hemolysin of the disclosure attached to a cargo.

In an aspect, a method of translocating a cargo across a lipid membrane is provided, the method comprising, contacting a lipid membrane with a α-Hemolysin attached to the cargo.

In an aspect, a method of delivering a therapeutic peptide to a cell, the method comprising contacting the cell with α-Hemolysin attached to the therapeutic peptide. In an aspect, a method of delivering a therapeutic peptide to a cell, the method comprising contacting the cell with α-Hemolysin attached to a nucleic acid encoding the therapeutic peptide.

In an aspect, a method of delivering an antibody for inhibiting protein-protein-interaction to a cell, the method comprising contacting the cell with a α-Hemolysin attached to an antibody for inhibiting protein-protein-interaction. In an aspect, a method of delivering an antibody for inhibiting protein-protein-interaction to a cell, the method comprising contacting the cell with a α-Hemolysin attached to a nucleic acid encoding an antibody for inhibiting protein-protein-interaction.

In an aspect, a method of delivering an enzyme to a cell, the method comprising contacting the cell with a α-Hemolysin attached to an enzyme. In an aspect, a method of delivering an enzyme to a cell, the method comprising contacting the cell with a α-Hemolysin attached to a nucleic acid encoding an enzyme.

In an aspect, a method of up- or downregulation of gene expression in a cell, comprising contacting the cell with a α-Hemolysin attached to a transcription factor. In an aspect, a method of up- or downregulation of gene expression in a cell, comprising contacting the cell with a α-Hemolysin attached to a nucleic acid encoding a transcription factor.

In an aspect, a method of editing the genome of a cell is provided, the method comprising contacting the cell with a α-Hemolysin attached to a CRISPR nuclease. In an aspect, a method of editing the genome of a cell is provided, the method comprising contacting the cell with a α-Hemolysin attached to a nucleic acid encoding a CRISPR nuclease.

In an aspect, a method of killing a tumor cell is provided, the method comprising contacting the cell with a α-Hemolysin attached to a cytotoxic molecule.

In an aspect, a method to functionalize the extracellular membrane side of a lipid vesicle is provided, the method comprising expressing a α-Hemolysin with a functionalization insertion in a lipid vesicle.

In an aspect, a method to functionalize the extracellular membrane side of a lipid vesicle is provided, the method comprising encapsulating a α-Hemolysin with a functionalization insertion in a lipid vesicle.

In an aspect, a method to functionalize the surface of an artificial cell-cell adhesion system is provided, the method comprising expressing one or more α-Hemolysin comprising cell adhesion molecules in an artificial cell.

In an aspect, a method to functionalize the surface of an artificial cell-cell adhesion system is provided, the method comprising encapsulating one or more α-Hemolysin comprising cell adhesion molecules in an artificial cell.

In an aspect, a method of displaying polypeptides on the surface of an artificial cell is provided, the method comprising expressing a α-Hemolysin with a loop region comprising a polypeptide insert in an artificial cell.

In an aspect, a method of displaying polypeptides on the surface of an artificial cell is provided, the method comprising encapsulating a α-Hemolysin with a loop region comprising a polypeptide insert in an artificial cell.

In an aspect, a method to functionalize the intracellular membrane side of a lipid vesicle is provided, the method comprising contacting the lipid vesicle with a α-Hemolysin.

In an aspect, a method to functionalize the inside surface of an artificial cell-cell adhesion system is provided, the method comprising contacting the artificial cell with one or more α-Hemolysin comprising cell adhesion molecules.

In an aspect, a method of displaying polypeptides on the inside surface of an artificial cell is provided, the method comprising contacting the artificial cell with a α-Hemolysin with a loop region comprising a polypeptide.

In an aspect, a method of screening a peptide-library by peptide display is provided, the method comprising contacting a plurality of cells with a plurality of α-Hemolysins attached to a library of peptides, contacting the library of peptides with a target molecule under conditions that allow the target molecule to bind the peptide library, removing cells not bound to the target molecule, identifying peptide binders to the target molecule.

In an aspect, a method of screening a peptide-library by peptide display is provided, the method comprising expressing plurality of α-Hemolysins attached to a library of peptides in a plurality of cells, contacting the library of peptides with a target molecule under conditions that allow the target molecule to bind the peptide library, removing cells not bound to the target molecule, identifying peptide binders to the target molecule.

### VI. Kits

In an aspect is provided kits comprising any of the compositions or compounds described above. In aspects the compositions and compounds are combined. In aspects, the kit further includes any of the agents described above. In aspects, one or more of the compounds or agents are in a separate container. The kits can further include instructions for preparation and use.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: α-Hemolysin Loop Insertions and α-Hemolysin Pore Formation

This example describes the design and characterization of loop variant α-Hemolysins and determination of pore formation by the variant α-Hemolysins.

It is known that the loop tolerates the insertion of 5 Histidines. Briefly, a 6×His-tag flanked by two flexible GGGGS-linkers was inserted between residues D128 and K131 into the α-Hemolysin-loop (FIG. 1A) of WT α-Hemolysin (αHL) SEQ ID NO: 3. Residues T129 and G130 of WT α-Hemolysin were deleted.

Briefly, gene constructs were cloned into the pTNT™ vector (Promega). BL21(DE3) Competent E. coli cells (NEB) were transformed with the respective plasmid. 300 ml of LB-medium with carbenicillin (100 μg/ml) was inoculated with a single colony and incubated on a shaker at 37° C. overnight. Cells were collected through centrifugation for 5 minutes at 5000 g. The resulting cell pellet was resuspended in 3 ml lysis buffer (HEPES 20 mM pH=7.5, NaCl 500 mM, imidazole 10 mM, PMSF 1 mM). Cells were lysed by sonication on ice (3 s on, 3 s off, 10 min, 50% amplitude). The lysate was centrifuged (15.000×g, 30 min, 4° C.) and filtered through a 0.45 μm syringe filter. The resulting cleared solution was applied to a gravity Ni-NTA column, loaded with 1 ml HisPur™ Ni-NTA Resin that had been pre-equilibrated with lysis buffer. After incubation with the lysate on an overhead spinner at 4° C. for 30 min, the column was washed 4 times with wash buffer (HEPES 20 mM pH=7.5, NaCl 500 mM, imidazole 30 mM; 1 ml each wash). Protein was eluted into 4 volumes of elution buffer (HEPES 20 mM pH=7.5, NaCl 500 mM, imidazole 300 mM; 0.5 ml each elution fraction). Protein fractions were identified by SDS-PAGE and the relevant fractions pooled and dialyzed against storage buffer (HEPES 20 mM pH=7.5, NaCl 500 mM). Proteins were stored at 4° C. for several days or at −80° C. for long-term storage.

The variant α-Hemolysins were characterized in pore formation assays to determine if the loop tolerates inserts larger than 5 amino acids and to insure increased accessibility of the His-tag after membrane translocation.

Briefly, GUVs were formed through the inverse emulsion method. Briefly, a 1 mM lipid stock (DOPC 60 mol %, cholesterol 40 mol %) in mineral oil was prepared. To 100 μl of this lipid solution was added 10 μl of encapsulation solution, containing all the components to be encapsulated into GUVs. This mixture was emulsified by vortexing. The resulting emulsion was layered on top of lower buffer solution. If not specified otherwise, this lower buffer solution comprised of 20 mM HEPES pH=7.0, NaCl 500 mM. This two-layer system was subjected to centrifugation at 10.000×g for 10 minutes. The oil layer was removed and the GUV pellet was resuspended in 20 μl lower buffer. For forming GUVs encapsulating a PURExpress® expression system, this GUV formation protocol was scaled down by half: 50 μl lipid solution, 5 μl encapsulation solution, 50 μl lower buffer solution.

While not wishing to be held by theory, the addition of a flexible linker can make the His-tag more accessible by increasing the distance from the membrane and reducing steric interaction. A α-Hemolysin with a C-terminal GFP fusion and a 6×His-tag in the loop flanked by two linkers was generated. Upon treatment of Cy5-encapsulating GUVs the protein showed both GFP localization to the membrane (FIG. 1B) and Cy5 leakage (FIG. 1C), indicating the formation of a fully functional pore.

### Example 2: α-Hemolysin Loop Translocation Through a Membrane and Loop Accessibility

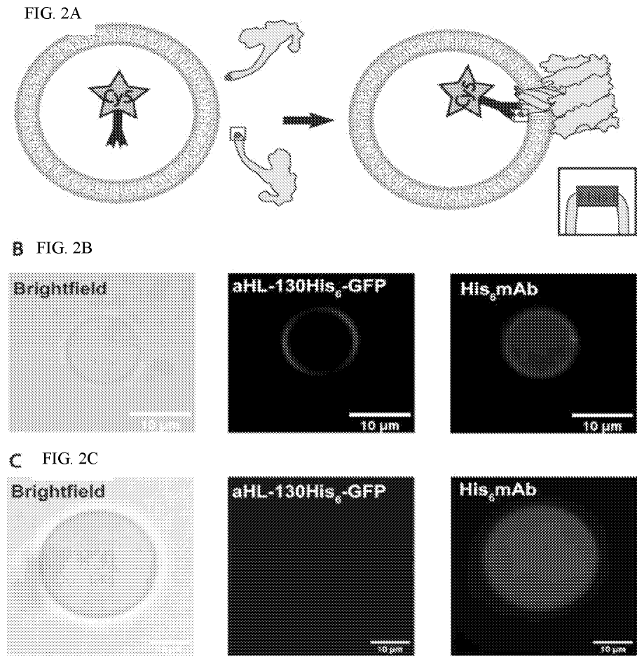

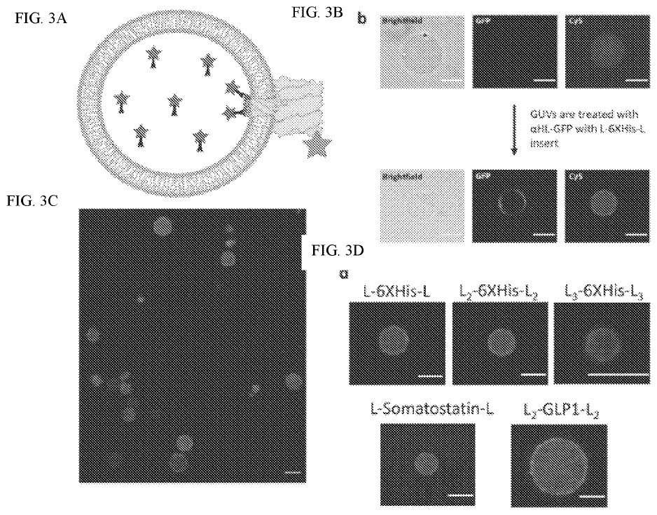

To show that the His-tag was fully translocated and accessible from the other membrane side the pore formation experiment in Example 1 was repeated with GUVs encapsulating a Cy5-conjugated anti-His-tag antibody (FIG. 2A). Because the antibody is too big it cannot diffuse through the α-Hemolysin pores. Upon treatment with the α-Hemolysin fusion protein, the antibody was localized to the GUV membrane (FIG. 2B), giving further proof that the His-tag gets fully translocated through the membrane and can be bound by an antibody. In comparison, untreated GUVs encapsulating Cy5-conjugated anti-His-tag antibody do not show membrane localization of the antibody over time. (FIG. 2C).

### Example 3: Large α-Hemolysin Loop Insertions

Longer peptide inserts were introduced into the loop. Longer length linkers were examined first. Both 6×His inserts with a (GGGGS)₂-linker and a (GGGGS)₃-linker formed functional pores with the His-tag being accessible to an anti-His-tag antibody. Different peptide inserts were investigated. Insertion of an E2-epitope tag into the α-Hemolysin loop did not inhibit pore formation as demonstrated by inducing Cy5 leakage from GUVs. In addition, insertion of the cyclic peptide Somatostatin-14 into the loop demonstrated translocation of the peptide both through antibody binding and Cy5 leakage. (Table 4)

### Vesicle Leakage Assay

Briefly, GUVs were formed as described with encapsulation solution containing Cy5 (1 μM) and 3.5% (wt/vol) Ficoll® 400 in buffer (20 mM HEPES pH=7.0, NaCl 500 mM). The resulting GUVs were treated with protein to a final protein concentration of 10 μM. After incubation on an overhead spinner at room temperature for 2 h, the GUVs were imaged on a confocal microscope.

TABLE 4
Amino acid sequences which could be inserted into α-Hemolysin loop without
disrupting its activity.

| Insert | Amino Acid Sequence | Length (Amino Acids) |
|---|---|---|
| I-6XHis-I | GGGGS HHHHHH GGGGS | 16 |
| I₂-6XHis-I₂ | (GGGGS)₂ HHHHHH (GGGGS)₂ | 26 |
| I₃-6XHis-I₃ | (GGGGS)₃ HHHHHH (GGGGS)₃ | 36 |
| I-E2-I | GGGGS SSTSSDFRDR GGGGS | 20 |
| Somatostatin-14 | GGGGS AGCKNFFWKTFTSC GGGGS | 24 |

Increasing the insert size to 50 amino acids with the peptide sequence of Glucagon-like Peptide-1 led to a non-functional protein. The same was true for inserting large proteins like the SNAP-tag, GFP, or the archaeal 7 kDa DNA-binding protein Sac7e.

Apart from size, charge also reduced the potential for forming a functional α-Hemolysin: Inserting two different, 41 amino acid, leucine-zipper peptides (LZA and LZB) led to inactive proteins. In order to further investigate the influence of highly charged peptides, the inserts were made shorter while keeping the highly charged nature of leucine zipper peptides. The artificial leucine zipper peptide pair CPA and CPB also yielded inactive protein at a peptide length of 35 amino acids. Peptides with 5 alternating charged amino acids (Lysine and glutamic acid) separated by glycine spacers were examined. These short, 23 amino acid inserts (CPC and CPD) likewise yielded protein with no lytic activity. (Table 5)

TABLE 5
Amino acid sequences which could not be inserted into Hemolysin loop without
disrupting its activity

| | | Length |
|---|---|---|
| | | (Amino |
| Insert | Amino Acid Sequence | Acids) |
| I-LZA-I | GGGGS AQLKKKLQALKKKNAQLKWKLQALKKKLAQK GGGGS | 41 |
| I-LZB-I | GGGGS AQLEKELQALEKENAQLEWELQALEKELAQK GGGGS | 41 |
| I-CPA-I | GGGGS GEIAALEKEIAALEWEIAALEQGS GGGGS | 34 |
| I-CPB-I | GGGGS GKIAALKYKIAALKKKIAALKQGS GGGGS | 34 |
| I-CPC-I | GGGGS EGGKGGEGGKGGE GGGGS | 23 |
| I-CPD-I | GGGGS KGGEGGKGGEGGK GGGGS | 23 |
| GLP1 | (GGGGS)₂ HAEGTFTSDVSSYLEGQAAKEFIAWLVKGR (GGGGS)₂ | 50 |
| Sac7e | GGGGS | 75 |
| | MAKVRFKYKGEEKEVDTSKIKKVWRVGKMVSFTYDDNGKTGRGAVS | |
| | EKDAPKELMDMLARAEKKK | |
| | GGGGS | |
| I-SNAP-I | GGGGS | 192 |
| | MDKDCEMKRTTLDSPLGKLELSGCEQGLHRIIFLGKGTSAADAVEVPA | |
| | PAAVLGGPEPLMQATAWLNAYFHQPEAIEEFPVPALHHPVFQQESFT | |
| | RQVLWKLLKVVKFGEVISYSHLAALAGNPAATAAVKTALSGNPVPILIP | |
| | CHRVVQGDLDVGGYEGGLAVKEWLLAHEGHRLGKPGL | |
| | GGGGGS | |
| I-GFP-I | GGGGS | 248 |
| | MRKGEELFTGVVPILVELDGDVNGHKFSVRGEGEGDATNGKLTLKFIC | |
| | TTGKLPVPWPTLVTTLTYGVQCFARYPDHMKQHDFFKSAMPEGYVQ | |
| | ERTISFKDDGTYKTRAEVKFEGDTLVNRIELKGIDFKEDGNILGHKLEYN | |
| | FNSHNVYITADKQKNGIKANFKIRHNVEDGSVQLADHYQQNTPIGDG | |
| | PVLLPDNHYLSTQSVLSKDPNEKRDHMVLLEFVTAAGITHGMDELYK | |
| | GGGGS | |

### Example 4: Peptide-Translocation by α-Hemolysin for Generation of Functionalized GUVs and Tissue-Like Structures

Artificial cells can be created which are able to express, translocate and display a short peptide on the cell surface, thereby gaining the ability to interact with the extracellular environment.

A PURExpress system was encapsulated into GUVs for expression of α-Hemolysin with the 1-6×His-1 insert. In order to show that these GUVs display a His-tag, a second population of GUVs for which the membrane was doped with 2% 18:1 DGS-NTA(Ni) was created.

Briefly, GUVs expressing either αHL with the K3 insert or the E3 insert were formed as described with the encapsulation solution containing a PURExpress® expression mix prepared according to the manufacturer's instructions. Two different batches of GUVs were prepared. For GUVs expressing αHL with the K3 insert, to 5 μl PURExpress® mix was added mCherry to a final concentration of 1 μM, plasmid coding for αHL with the K3 insert (75 ng), and 3.5% (wt/vol) Ficoll® 400. For GUVs expressing αHL with the E3 insert, to 5 μl PURExpress® mix was added CFP to a final concentration of 1 M, plasmid coding for αHL with the E3 insert (75 ng), and 3.5% (wt/vol) Ficoll® 400. To account for the high protein concentration in the PURExpress® mix, the lower buffer solution was changed to 50 mM Tris pH=7.0, alanine 100 mM, BSA (66 μM). The GUV pellet was resuspended in expression buffer (PURExpress® solution A (2.0 μl), H2O (3.0 μl)).

GUVs expressing αHL with the K3 insert and GUVs expressing αHL with the E3 insert were mixed at a ratio of 1:1 and incubated on an overhead rotator at 37° C. for 2 hours to induce protein expression and subsequently aggregation into tissue-like structures. The resulting tissue-like structures were imaged by confocal microscopy.

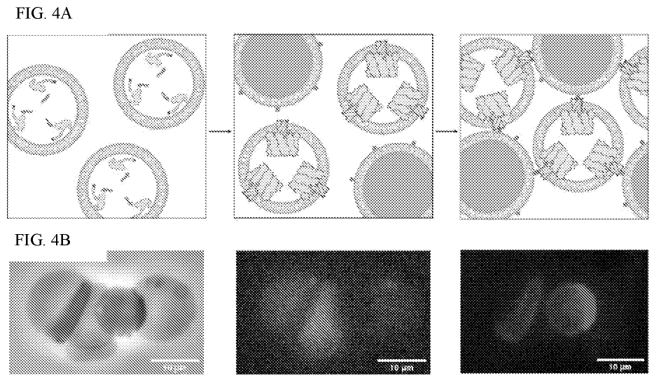

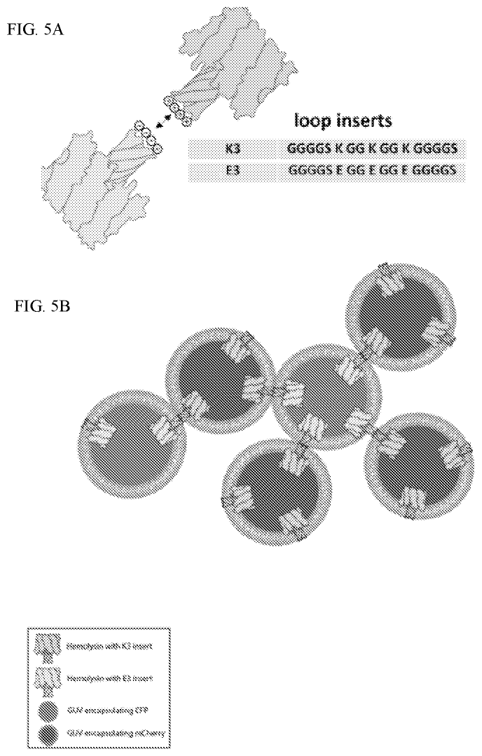

Upon mixing, two populations of GUVs showed strong GUV-GUV-interactions which led to the formation of tissue-like structures. (FIGS. 4A and 4B and FIG. 5A-5C)

### Hydrogen Peroxide Signaling in Artificial Tissues

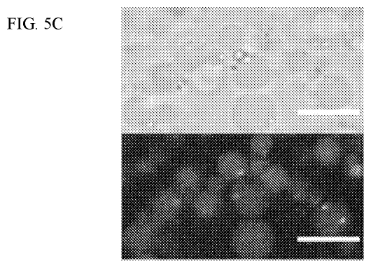

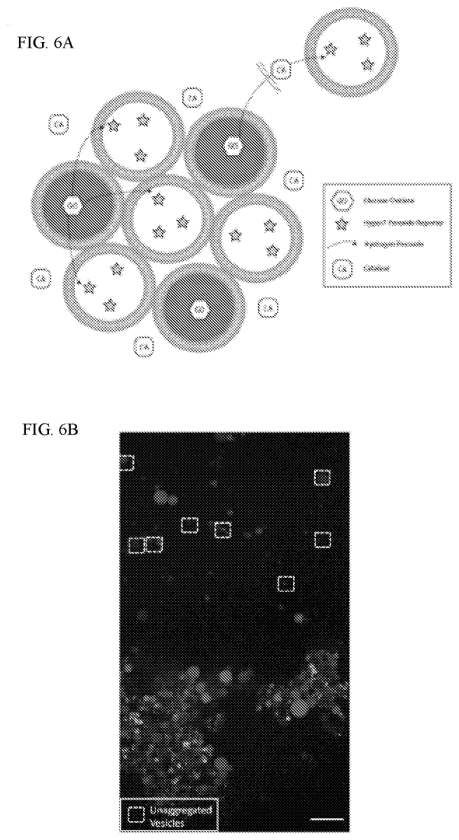

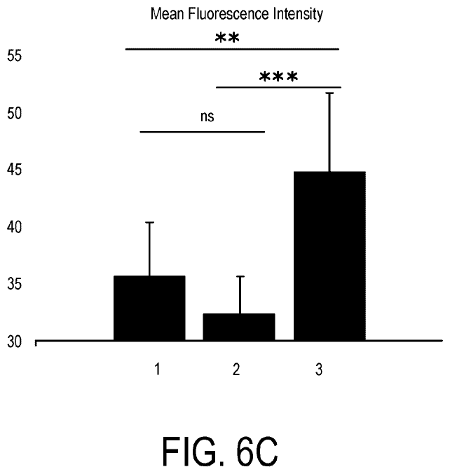

For the formation of sender and receiver cells the above protocol for artificial tissue formation was modified slightly: For sender GUVs, to 5 μl PURExpress® mix was added mCherry to a final concentration of 1 μM, plasmid coding for αHL with the K3 insert (75 ng), glucose oxidase (1 U/ml) and 3.5% (wt/vol) Ficoll® 400. For receiver GUVs, to 5 μl PURExpress® mix was added plasmid coding for αHL with the E3 insert (75 ng), 3.5% (wt/vol) Ficoll® 400 and Hyper7 to a final concentration of 2 μM. A 1:1 mixture of sender and receiver GUVs were supplemented with 1% (wt/vol) glucose and catalase to a final concentration of 3 U/ml. The resulting mixture was incubated on an overhead rotator at 37° C. for 2 hours to induce protein expression, aggregation into tissue-like structures and hydrogen peroxide signaling. The formed tissue-like structures were imaged by confocal microscopy. (FIGS. 6A to 6C)

### Statistics and Reproducibility

Statistically significant differences in FIG. 6c are indicated based on an independent t-test (two-tailed): ***P<0.001; **P<0.01; NS, not significant. Specifically, 1 (GUVs in artificial tissue in the absence of glucose) vs 2 (GUVs outside of artificial tissues in the presence of glucose) P=0.0830. 1 (GUVs in artificial tissue in the absence of glucose) vs 3 (GUVs in artificial tissue in the presence of glucose) P=0.00351. 2 (GUVs outside of artificial tissues in the presence of glucose) vs 3 (GUVs in artificial tissue in the presence of glucose) P=0.000210.

### Example 5: α-Hemolysin Bioconjugates and Translocation of Cargo in GUVs

Hemolysin was cloned with a single cysteine in the loop, flanked by GGGGS linkers. Insert I-C-I:

| | | |
|---|---|---|
| I-C-I | GGGGS C GGGGS | 11 amino acids in length |

Since the wildtype α-Hemolysin sequence does not contain any cysteine, this 130C-Hemolysin can be used for site specific conjugation of other biomolecules based on maleimide chemistry.

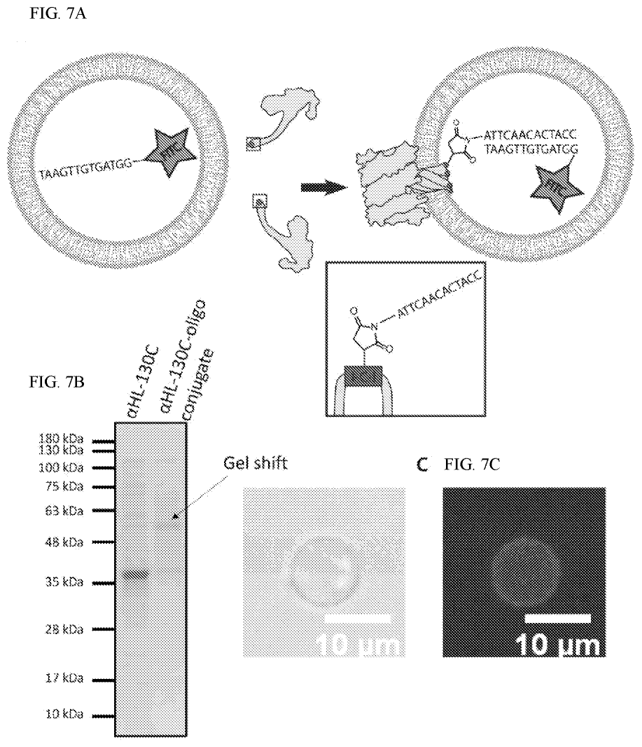

A 5′-Maleimide-DNA(7 mer)-FITC-3′ oligo was conjugated to the protein. The resulting conjugate showed strong membrane binding and was able to induce leakage of Cy5 from GUVs. Increasing lengths of oligonucleotide were tested. Conjugates with a 13 mer and 30 mer oligo also showed membrane binding and Cy5 leakage. To further prove translocation of the oligo, the 13 mer conjugate without a FITC-label was prepared. (FIG. 7B) A complementary 13 mer oligo with a FITC-label was encapsulated into GUVs. Treatment of these GUVs with the conjugate led to hybridization of the oligos, shown by increased FITC membrane fluorescence, which indicates translocation of the conjugated oligo. (FIG. 7C)

For the larger 30 mer oligo, dilution of the conjugate with unconjugated 130C-Hemolysin improved translocation across the membrane. It is hypothesized that this is because the heptameric pore does not tolerate 7 large oligos, but can tolerate a smaller amount, meaning that by dilution with unconjugated 130C-Hemolysin, formation of hetero-heptameric α-Hemolysin pores, comprised of conjugated and unconjugated monomers is possible.

Dilution with wild-type α-Hemolysin or 130C-Hemolysin for some of the larger protein inserts were examined. Dilution with wild-type α-Hemolysin did not lead to improved membrane binding. However, dilution with 130C-Hemolysin led to both membrane binding and Cy5-leakage. The resulting system enables endocytosis-independent delivery of biological cargos.

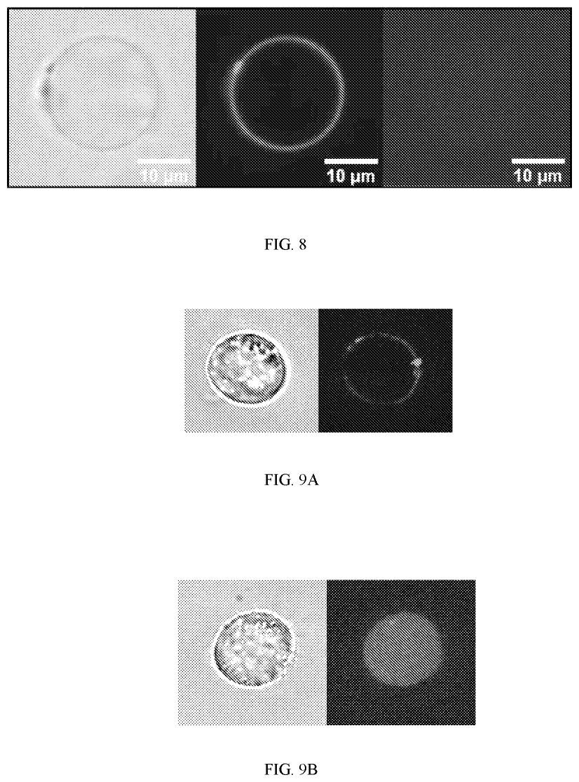

Furthermore, the 130C-Hemolysin variant translocated a whole SNAP-tag-GFP fusion (47 kDa), protein across a membrane. (FIG. 8).

TABLE 6
130C-Hemolysin was reacted with BG-maleimide. The conjunction to BG makes
the protein reactive to SNAP-tagged proteins. Reaction with SNAP-GFP
gave a α-Hemolysin with insert 1-C(SNAP-GFP)-1.

| Insert | Amino Acid Sequence | (Amino |
|---|---|---|
| 1-C(SNAP-GFP)-1 | MDKDCEMKRTTLDSPLGKLELSGCEQGLHRIIFLGKGTS | 440 |
| | AADAVEVPAPAAVLGGPEPLMQATAWLNAYFHQPEAI | |
| | EEFPVPALHHPVFQQESFTRQVLWKLLKVVKFGEVISYS | |
| | HLAALAGNPAATAAVKTALSGNPVPILIPCHRVVQGDLD | |
| | VGGYEGGLAVKEWLLAHEGHRLGKPGL | |
| | MRKGEELFTGVVPILVELDGDVNGHKFSVRGEGEGDAT | |
| | NGKLTLKFICTTGKLPVPWPTLVTTLTYGVQCFARYPDH | |
| | MKQHDFFKSAMPEGYVQERTISFKDDGTYKTRAEVKFE | |
| | GDTLVNRIELKGIDFKEDGNILGHKLEYNFNSHNVYITAD | |
| | KQKNGIKANFKIRHNVEDGSVQLADHYQQNTPIGDGPV | |
| | LLPDNHYLSTQSVLSKDPNEKRDHMVLLEFVTAAGITHG | |
| | MD ELYK | |

### Example 6: α-Hemolysin Bioconjugates and Translocation of Cargo in Cells

This example describes the membrane insertion and translocation of a α-Hemolysin conjugated to a DNA FITC labeled oligonucleotide (αHL-130C 5′-Maleimide-DNA (22 mer)-FITC-3′, αHL-130C-[Mal]-TAGCTTATCAGACTGATGTTGA-[FITC]).

Briefly, the FITC labeled αHL was prepared as described in Example 5. THP1 cells were incubated with the FITC labeled αHL, and the resulting fluorescence was determined after 5 min and 30 min. The results show, that after 5 min the cellular membrane displays FITC fluorescence. After 30 min incubation, the FITC fluorescence on the cell is uniformly distributed The results show, that the FITC labeled αHL was translocated through the cellular membrane.

### REFERENCES

Sunwoo Koo, Stephen Cheley, and Hagan Bayley, ACS Central Science 2019 5 (4), 629639

### REFERENCES

Akiyama, S. K., Nagata, K. & Yamada, K. M. Cell surface receptors for extracellular matrix components. Biochimica et Biophysica Acta (BBA)—Reviews on Biomembranes 1031, 91-110 (1990).
Bachmann, M., Kukkurainen, S., Hytonen, V. P. & Wehrle-Haller, B. Cell Adhesion by Integrins. Physiological Reviews 99, 1655-1699 (2019).
Cymer, F., von Heijne, G. & White, S. H. Mechanisms of Integral Membrane Protein Insertion and Folding. Journal of Molecular Biology 427, 999-1022 (2015).
Seddon, A. M., Cumow, P. & Booth, P. J. Membrane proteins, lipids and detergents: not just a soap opera. Biochimica et Biophysica Acta (BBA)—Biomembranes 1666, 105-117 (2004).
Noireaux, V. & Libchaber, A. A vesicle bioreactor as a step toward an artificial cell assembly. Proceedings of the National Academy of Sciences 101, 17669-17674 (2004).
Dinges, M. M., Orwin, P. M. & Schlievert, P. M. Exotoxins of Staphylococcus aureus. Clinical Microbiology Reviews 13, 16-34 (2000).
Deamer, D., Akeson, M. & Branton, D. Three decades of nanopore sequencing. Nat Biotechnol 34, 518-524 (2016).
Walker, B. & Bayley, H. Key Residues for Membrane Binding, Oligomerization, and Pore Forming Activity of Staphylococcal α-Hemolysin Identified by Cysteine Scanning Mutagenesis and Targeted Chemical Modification (*). Journal of Biological Chemistry 270, 23065-23071 (1995).
Palmer, M., Jursch, R., Weller, U., Valeva, A., Hilgert, K, Kehoe, M. & Bhakdi, S. Staphylococcus aureus alpha-toxin. Production of functionally intact, site-specifically modifiable protein by introduction of cysteine at positions 69, 130, and 186. Journal of Biological Chemistry 268, 11959-11962 (1993).
Valeva, A. et al. Transmembrane β-barrel of staphylococcal α-toxin forms in sensitive but not in resistant cells. Proceedings of the National Academy of Sciences 94, 11607-11611 (1997).
Ward, R. J., Palmer, M., Leonard, K. & Bhakdi, S. Identification of a Putative Membrane-Inserted Segment in the .alpha.-Toxin of Staphylococcus aureus. Biochemistry 33, 7477-7484 (1994).
Krasilnikov, O. V., Capistrano, M.-F. P., Yuldasheva, L. N. & Nogueira, R. A. Influence of Cys-130 S. aureus Alpha-toxin on Planar Lipid Bilayer and Erythrocyte Membranes. J. Membrane Biol. 156, 157-172 (1997).
Krishnasastry, M., Walker, B., Braha, O. & Bayley, H. Surface labeling of key residues during assembly of the transmembrane pore formed by staphylococcal α-hemolysin. FEBS Letters 356, 66-71 (1994).
Valeva, A. et al. Membrane Insertion of the Heptameric Staphylococcal α-Toxin Pore: A DOMINO-LIKE STRUCTURAL TRANSITION THAT IS ALLOSTERICALLY MODULATED BY THE TARGET CELL MEMBRANE *. Journal of Biological Chemistry 276, 14835-14841 (2001).
Walker, B., Kasianowicz, J., Krishnasastry, M. & Bayley, H. A pore-forming protein with a metal-actuated switch. Protein Engineering. Design and Selection 7, 655-662 (1994).
Deshpande, S., Caspi, Y., Meijering, A. E. C. & Dekker, C. Octanol-assisted liposome assembly on chip. Nat Commun 7, 10447 (2016).
Saito, G., Swanson, J. A. & Lee, K.-D. Drug delivery strategy utilizing conjugation via reversible disulfide linkages: role and site of cellular reducing activities. Advanced Drug Delivery Reviews 55, 199-215 (2003).
Russo, M. J., Bayley, H. & Toner, M. Reversible permeabilization of plasma membranes with an engineered switchable pore. Nat Biotechnol 15, 278-282 (1997).
M. Thomas, J., S. Friddin, M., Ces, O. & Elani, Y. Programming membrane permeability using integrated membrane pores and blockers as molecular regulators. Chemical Communications 53, 12282-12285 (2017).
Wadia, J. S. & Dowdy, S. F. Protein transduction technology. Current Opinion in Biotechnology 13, 52-56 (2002).

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. The scope of the invention is thus indicated by the appended claims rather than by the foregoing description, and all changes that come within the meaning and range of equivalency of the claims are intended to be embraced therein.

### Embodiments

Embodiment 1. An α-Hemolysin polypeptide comprising an amino acid insert sequence between amino acids corresponding to positions D128 and K131 of SEQ ID NO:3, wherein the amino acid insert sequence is 5 to 70 amino acids in length.
Embodiment 2. The α-Hemolysin polypeptide of embodiment 1, wherein the α-Hemolysin polypeptide without said amino acid insert sequence has at least 80%, 85%, 90%, 91%, 92%, 93%, 94%, 95%, 96%, 97%, 98%, or 99% sequence identity to SEQ ID NO: 3.
Embodiment 3. The α-Hemolysin polypeptide of embodiment 1 or embodiment 2, wherein the amino acid sequence between amino acids corresponding to D128 and K131 of SEQ ID NO:3 comprises an amino acid deletion.
Embodiment 4. The α-Hemolysin polypeptide of any one of embodiments 1 to 3, wherein the α-Hemolysin polypeptide comprises a cysteine mutation corresponding to a position of amino acid 130 of SEQ ID NO:3.
Embodiment 5. The α-Hemolysin polypeptide of any one of embodiments 1 to 4, wherein the α-Hemolysin polypeptide comprises a histidine to leucine mutation corresponding to amino acid H48 of SEQ ID NO:3.
Embodiment 6. The α-Hemolysin polypeptide of any one of embodiments 1 to 5, wherein the α-Hemolysin polypeptide comprises a deletion corresponding to amino acids D2-G23 of SEQ ID NO:3.
Embodiment 7. The α-Hemolysin polypeptide of any one of embodiments 1 to 6, wherein the amino acid insert sequence is less than 41 amino acids in length.
Embodiment 8. The α-Hemolysin polypeptide of any one of embodiments 1 to 6, wherein the amino acid insert sequence is less than 52 amino acids in length.
Embodiment 9. The α-Hemolysin polypeptide of any one of embodiments 1 to 8, wherein the amino acid insert sequence comprises a cysteine.
Embodiment 10. The α-Hemolysin polypeptide of embodiment 1, wherein the amino acid insert sequence comprises a glycine linker.
Embodiment 11. The α-Hemolysin polypeptide of any one of embodiments 1 to 10, additionally comprising a bioconjugate reactive moiety.
Embodiment 12. The α-Hemolysin polypeptide of embodiment 11, wherein the bioconjugate reactive moiety is attached to an amino acid of the amino acid insert sequence.
Embodiment 13. The α-Hemolysin polypeptide of any one of embodiments 1 to 12, additionally comprising a cargo molecule.
Embodiment 14. The α-Hemolysin polypeptide of embodiment 13, wherein the cargo molecule is attached to the amino acid insert sequence.
Embodiment 15. The α-Hemolysin polypeptide of any one of embodiments 12 to 14, wherein the cargo molecule is covalently attached to a cysteine in the amino acid insert sequence.
Embodiment 16. The α-Hemolysin polypeptide any one of embodiments 12 to 14, wherein the cargo molecule is covalently attached by a bioconjugate to a cysteine in the amino acid insert sequence.
Embodiment 17. The α-Hemolysin polypeptide any one of embodiments 12 to 14, wherein the cargo molecule is selected from the group of a polynucleotide, a polypeptide, a lipid, a carbohydrate, or a small molecule.
Embodiment 18. A phospholipid membrane comprising the α-Hemolysin polypeptide of any one of embodiments 1 to 17.
Embodiment 19. The phospholipid membrane of embodiment 18, further comprising a macromolecule.
Embodiment 20. The phospholipid membrane of embodiment 19, wherein the macromolecule is a polypeptide or polynucleotide.
Embodiment 21. The phospholipid membrane of embodiment 20, wherein the polynucleotide is DNA or RNA.
Embodiment 22. The phospholipid membrane of embodiment 20, wherein the polynucleotide is mRNA, siRNA, miRNA, or microRNA.
Embodiment 23. The phospholipid membrane any one of embodiments 18 to 22, wherein the macromolecule comprises a bioconjugate reactive moiety.
Embodiment 24. The phospholipid membrane of embodiment 23, wherein the bioconjugate reactive moiety is a maleimide.
Embodiment 25. The phospholipid membrane any one of embodiments 18 to 22, wherein the macromolecule comprises a bioconjugate.
Embodiment 26. A tissue-like structure comprising a plurality of phospholipid membranes of any one of embodiments 18 to 25.
Embodiment 27. A peptide of the formula:

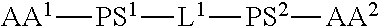

wherein
PS¹ is an amino acid sequence having at least 90% sequence identity to SEQ ID: 35;
PS² is an amino acid sequence having at least 90% sequence identity to SEQ ID: 36;
   L¹ is a peptidyl linker having 1-70 amino acids; and
AA¹ and AA² are independently hydrogen or amino acid sequences having 1-100 amino acids.
Embodiment 28. The peptide of embodiment 27, wherein PS¹ is SEQ ID: 34.
Embodiment 29. The peptide of embodiment 27, wherein the PS¹ comprises a Histidine to Leucine mutation corresponding to a position of amino acid H25 of SEQ ID NO:35.
Embodiment 30. The peptide of any one of embodiments 27 to 29, wherein L¹ is selected from the amino acid sequences in Table 2.
Embodiment 31. The peptide of embodiment 27, wherein L¹ comprises a cysteine residue.
Embodiment 32. The peptide of embodiment 27, wherein L¹ is less than 41 amino acids in length.
Embodiment 33. The peptide of embodiment 27, wherein L¹ is less than 52 amino acids in length.
Embodiment 34. The peptide any one of embodiments 27 to 33, wherein L¹ comprises a glycine linker.
Embodiment 35. The peptide any one of embodiments 27 to 34, wherein AA¹ and/or AA² are a fusion protein.
Embodiment 36. The peptide any one of embodiments 27 to 35, additionally comprising a bioconjugate reactive moiety.
Embodiment 37. The peptide of embodiment 27, wherein the bioconjugate reactive moiety is attached to an amino acid in L¹.
Embodiment 38. The peptide of any one of embodiments 27 to 37, wherein the peptide additionally comprises a cargo molecule.
Embodiment 39. The peptide of embodiment 38, wherein the cargo molecule is attached to L¹.
Embodiment 40. The peptide of embodiment 39, wherein the cargo molecule is covalently attached to a cysteine in L¹.
Embodiment 41. The peptide of embodiment 39, wherein the cargo molecule is covalently attached by a bioconjugate to a cysteine in L¹.
Embodiment 42. The peptide of embodiment 38, wherein the cargo molecule is selected from the group of a polynucleotide, a polypeptide, a lipid, a carbohydrate, or a small molecule.

SEQUENCE LISTING

| SEQ ID NO: | Description | SEQUENCE |
|---|---|---|
| 1 | WT-hemolysin | MKTRIVSSVTTTLLLGSILMNPVAGAADSDINIKTGTTDIGSNTTVKTG |
| | | DLVTYDKENGMHKKVFYSFIDDKNHNKKLLVIRTKGTIAGQYRVYS |
| | | EEGANKSGLAWPSAFKVQLQLPDNEVAQISDYYPRNSIDTKEYMSTL |
| | | TYGFNGNVTGDDTGKIGGLIGANVSIGHTLKYVQPDFKTILESPTDKK |
| | | VGWKVIFNNMVNQNWGPYDRDSWNPVYGNQLFMKTRNGSMKAA |
| | | DNFLDPNKASSLLSSGFSPDFATVITMDRKASKQQTNIDVIYERVRDD |
| | | YQLHWTSTNWKGTNTKDKWTDRSSERYKIDWEKEEMTN |
| 2 | Signal | MKTRIVSSVTTTLLLGSILMNPVAGAA |
| | Sequence | |
| 3 | Hemolysin | MDSDINIKTGTTDIGSNTTVKTGDLVTYDKENGMHKKVFYSFIDDKN |
| | without Signal | HNKKLLVIRTKGTIAGQYRVYSEEGANKSGLAWPSAFKVQLQLPDNE |
| | Sequence | VAQISDYYPRNSIDTKEYMSTLTYGFNGNVTGDDTGKIGGLIGANVSI |
| | | GHTLKYVQPDFKTILESPTDKKVGWKVIFNNMVNQNWGPYDRDSW |
| | | NPVYGNQLFMKTRNGSMKAADNFLDPNKASSLLSSGFSPDFATVITM |
| | | DRKASKQQTNIDVIYERVRDDYQLHWTSTNWKGTNTKDKWTDRSS |
| | | ERYKIDWEKEEMTN |
| 4 | hemolysin with | MDSDINIKTGTTDIGSNTTVKTGDLVTYDKENGMHKKVFYSFIDDKN |
| | Cysteine insert | HNKKLLVIRTKGTIAGQYRVYSEEGANKSGLAWPSAFKVQLQLPDNE |
| | | VAQISDYYPRNSIDTKEYMSTLTYGFNGNVTGDDGGGGSCGGGGSKI |
| | | GGLIGANVSIGHTLKYVQPDFKTILESPTDKKVGWKVIFNNMVNQN |
| | | WGPYDRDSWNPVYGNQLFMKTRNGSMKAADNFLDPNKASSLLSSG |
| | | FSPDFATVITMDRKASKQQTNIDVIYERVRDDYQLHWTSTNWKGTN |
| | | TKDKWTDRSSERYKIDWEKEEMTN |
| 5 | hemolysin with | MDSDINIKTGTTDIGSNTTVKTGDLVTYDKENGMHKKVFYSFIDDKN |
| | insert | HNKKLLVIRTKGTIAGQYRVYSEEGANKSGLAWPSAFKVQLQLPDNE |
| | | VAQISDYYPRNSIDTKEYMSTLTYGFNGNVTGDDinsertKIGGLIGANV |
| | | SIGHTLKYVQPDFKTILESPTDKKVGWKVIFNNMVNQNWGPYDRDS |
| | | WNPVYGNQLFMKTRNGSMKAADNFLDPNKASSLLSSGFSPDFATVIT |
| | | MDRKASKQQTNIDVIYERVRDDYQLHWTSTNWKGTNTKDKWTDRS |
| | | SERYKIDWEKEEMTN |
| 6 | hemolysin with | MDSDINIKTGTTDIGSNTTVKTGDLVTYDKENGMHKKVFYSFIDDKN |
| | 48H −> L | LNKKLLVIRTKGTIAGQYRVYSEEGANKSGLAWPSAFKVQLQLPDNE |
| | | VAQISDYYPRNSIDTKEYMSTLTYGFNGNVTGDDTGKIGGLIGANVSI |
| | | GHTLKYVQPDFKTILESPTDKKVGWKVIFNNMVNQNWGPYDRDSW |
| | | NPVYGNQLFMKTRNGSMKAADNFLDPNKASSLLSSGFSPDFATVITM |
| | | DRKASKQQTNIDVIYERVRDDYQLHWTSTNWKGTNTKDKWTDRSS |
| | | ERYKIDWEKEEMTN |
| 7 | hemolysin with | MDSDINIKTGTTDIGSNTTVKTGDLVTYDKENGMHKKVFYSFIDDKN |
| | 48H −> L insert | LNKKLLVIRTKGTIAGQYRVYSEEGANKSGLAWPSAFKVQLQLPDNE |
| | | VAQISDYYPRNSIDTKEYMSTLTYGFNGNVTGDDinsertKIGGLIGANV |
| | | SIGHTLKYVQPDFKTILESPTDKKVGWKVIFNNMVNQNWGPYDRDS |
| | | WNPVYGNQLFMKTRNGSMKAADNFLDPNKASSLLSSGFSPDFATVIT |
| | | MDRKASKQQTNIDVIYERVRDDYQLHWTSTNWKGTNTKDKWTDRS |
| | | SERYKIDWEKEEMTN |
| 8 | 48H −> L | MDSDINIKTGTTDIGSNTTVKTGDLVTYDKENGMHKKVFYSFIDDKN |
| | Cysteine Insert | LNKKLLVIRTKGTIAGQYRVYSEEGANKSGLAWPSAFKVQLQLPDNE |
| | | VAQISDYYPRNSIDTKEYMSTLTYGFNGNVTGDDGGGGSCGGGGSKI |
| | | GGLIGANVSIGHTLKYVQPDFKTILESPTDKKVGWKVIFNNMVNQN |
| | | WGPYDRDSWNPVYGNQLFMKTRNGSMKAADNFLDPNKASSLLSSG |
| | | FSPDFATVITMDRKASKQQTNIDVIYERVRDDYQLHWTSTNWKGTN |
| | | TKDKWTDRSSERYKIDWEKEEMTN |
| 9 | hemolysin with | MDLVTYDKENGMHKKVFYSFIDDKNHNKKLLVIRTKGTIAGQYRVY |
| | 2D-23G | SEEGANKSGLAWPSAFKVQLQLPDNEVAQISDYYPRNSIDTKEYMST |
| | truncated | LTYGFNGNVTGDDTGKIGGLIGANVSIGHTLKYVQPDFKTILESPTDK |
| | (amino acids | KVGWKVIFNNMVNQNWGPYDRDSWNPVYGNQLFMKTRNGSMKA |
| | 2D-23G are | ADNFLDPNKASSLLSSGFSPDFATVITMDRKASKQQTNIDVIYERVRD |
| | missing) | DYQLHWTSTNWKGTNTKDKWTDRSSERYKIDWEKEEMTN |
| 10 | hemolysin with | MDLVTYDKENGMHKKVFYSFIDDKNHNKKLLVIRTKGTIAGQYRVY |
| | 2D-23G | SEEGANKSGLAWPSAFKVQLQLPDNEVAQISDYYPRNSIDTKEYMST |
| | truncated | LTYGFNGNVTGDDinsertKIGGLIGANVSIGHTLKYVQPDFKTILESPTD |
| | (amino acids | KKVGWKVIFNNMVNQNWGPYDRDSWNPVYGNQLFMKTRNGSMK |
| | 2D-23G are | AADNFLDPNKASSLLSSGFSPDFATVITMDRKASKQQTNIDVIYERVR |
| | missing) with | DDYQLHWTSTNWKGTNTKDKWTDRSSERYKIDWEKEEMTN |
| | insert | |
| 11 | hemolysin with | MDLVTYDKENGMHKKVFYSFIDDKNHNKKLLVIRTKGTIAGQYRVY |
| | 2D-23G | SEEGANKSGLAWPSAFKVQLQLPDNEVAQISDYYPRNSIDTKEYMST |
| | truncated | LTYGFNGNVTGDDGGGGSCGGGGSKIGGLIGANVSIGHTLKYVQPDF |
| | (amino acids | KTILESPTDKKVGWKVIFNNMVNQNWGPYDRDSWNPVYGNQLFMK |
| | 2D-23G are | TRNGSMKAADNFLDPNKASSLLSSGFSPDFATVITMDRKASKQQTNI |
| | missing) with | DVIYERVRDDYQLHWTSTNWKGTNTKDKWTDRSSERYKIDWEKEE |
| | insert | MTN |
| 12 | hemolysin with | MDSDINIKTGTTDIGSNTTVKTGDLVTYDKENGMHKKVFYSFIDDKN |
| | L-6XHis-L | HNKKLLVIRTKGTIAGQYRVYSEEGANKSGLAWPSAFKVQLQLPDNE |
| | | VAQISDYYPRNSIDTKEYMSTLTYGFNGNVTGDD |
| | | GGGGSHHHHHHGGGGS |
| | | KIGGLIGANVSIGHTLKYVQPDFKTILESPTDKKVGWKVIFNNMVNQ |
| | | NWGPYDRDSWNPVYGNQLFMKTRNGSMKAADNFLDPNKASSLLSS |
| | | GFSPDFATVITMDRKASKQQTNIDVIYERVRDDYQLHWTSTNWKGT |
| | | NTKDKWTDRSSERYKIDWEKEEMTN |
| 13 | hemolysin with | MDSDINIKTGTTDIGSNTTVKTGDLVTYDKENGMHKKVFYSFIDDKN |
| | L₂-6XHis-L₂ | HNKKLLVIRTKGTIAGQYRVYSEEGANKSGLAWPSAFKVQLQLPDNE |
| | | VAQISDYYPRNSIDTKEYMSTLTYGFNGNVTGDDGGGGSGGGGSHH |
| | | HHHHGGGGSGGGGSKIGGLIGANVSIGHTLKYVQPDFKTILESPTDKK |
| | | VGWKVIFNNMVNQNWGPYDRDSWNPVYGNQLFMKTRNGSMKAA |
| | | DNFLDPNKASSLLSSGFSPDFATVITMDRKASKQQTNIDVIYERVRDD |
| | | YQLHWTSTNWKGTNTKDKWTDRSSERYKIDWEKEEMTN |
| 14 | hemolysin with | MDSDINIKTGTTDIGSNTTVKTGDLVTYDKENGMHKKVFYSFIDDKN |
| | L₃-6XHis-L₃ | HNKKLLVIRTKGTIAGQYRVYSEEGANKSGLAWPSAFKVQLQLPDNE |
| | | VAQISDYYPRNSIDTKEYMSTLTYGFNGNVTGDDGGGGSGGGGSGG |
| | | GGSHHHHHHGGGGSGGGGSGGGGSKIGGLIGANVSIGHTLKYVQPD |
| | | FKTILESPTDKKVGWKVIFNNMVNQNWGPYDRDSWNPVYGNQLFM |
| | | KTRNGSMKAADNFLDPNKASSLLSSGFSPDFATVITMDRKASKQQTN |
| | | IDVIYERVRDDYQLHWTSTNWKGTNTKDKWTDRSSERYKIDWEKEE |
| | | MTN |
| 15 | hemolysin with | MDSDINIKTGTTDIGSNTTVKTGDLVTYDKENGMHKKVFYSFIDDKN |
| | L- | HNKKLLVIRTKGTIAGQYRVYSEEGANKSGLAWPSAFKVQLQLPDNE |
| | Somatostatin-L | VAQISDYYPRNSIDTKEYMSTLTYGFNGNVTGDDGGGGSAGCKNFF |
| | | WKTFTSCGGGGSKIGGLIGANVSIGHTLKYVQPDFKTILESPTDKKVG |
| | | WKVIFNNMVNQNWGPYDRDSWNPVYGNQLFMKTRNGSMKAADNF |
| | | LDPNKASSLLSSGFSPDFATVITMDRKASKQQTNIDVIYERVRDDYQL |
| | | HWTSTNWKGTNTKDKWTDRSSERYKIDWEKEEMTN |
| 16 | hemolysin with | MDSDINIKTGTTDIGSNTTVKTGDLVTYDKENGMHKKVFYSFIDDKN |
| | L2-GLP1-L2 | HNKKLLVIRTKGTIAGQYRVYSEEGANKSGLAWPSAFKVQLQLPDNE |
| | | VAQISDYYPRNSIDTKEYMSTLTYGFNGNVTGDDGGGGSGGGGSHA |
| | | EGTFTSDVSSYLEGQAAKEFIAWLVKGRGGGGSGGGGSKIGGLIGAN |
| | | VSIGHTLKYVQPDFKTILESPTDKKVGWKVIFNNMVNQNWGPYDRD |
| | | SWNPVYGNQLFMKTRNGSMKAADNFLDPNKASSLLSSGFSPDFATVI |
| | | TMDRKASKQQTNIDVIYERVRDDYQLHWTSTNWKGTNTKDKWTDR |
| | | SSERYKIDWEKEEMTN |
| 17 | hemolysin with | MDSDINIKTGTTDIGSNTTVKTGDLVTYDKENGMHKKVFYSFIDDKN |
| | L-K3-L | HNKKLLVIRTKGTIAGQYRVYSEEGANKSGLAWPSAFKVQLQLPDNE |
| | | VAQISDYYPRNSIDTKEYMSTLTYGFNGNVTGDDGGGGSKGGKGGK |
| | | GGGGSKIGGLIGANVSIGHTLKYVQPDFKTILESPTDKKVGWKVIFNN |
| | | MVNQNWGPYDRDSWNPVYGNQLFMKTRNGSMKAADNFLDPNKAS |
| | | SLLSSGFSPDFATVITMDRKASKQQTNIDVIYERVRDDYQLHWTSTN |
| | | WKGTNTKDKWTDRSSERYKIDWEKEEMTN |
| 18 | hemolysin with | MDSDINIKTGTTDIGSNTTVKTGDLVTYDKENGMHKKVFYSFIDDKN |
| | L-E3-L | HNKKLLVIRTKGTIAGQYRVYSEEGANKSGLAWPSAFKVQLQLPDNE |
| | | VAQISDYYPRNSIDTKEYMSTLTYGFNGNVTGDDGGGGSEGGEGGE |
| | | GGGGSKIGGLIGANVSIGHTLKYVQPDFKTILESPTDKKVGWKVIFNN |
| | | MVNQNWGPYDRDSWNPVYGNQLFMKTRNGSMKAADNFLDPNKAS |
| | | SLLSSGFSPDFATVITMDRKASKQQTNIDVIYERVRDDYQLHWTSTN |
| | | WKGTNTKDKWTDRSSERYKIDWEKEEMTN |
| 19 | hemolysin with | MDSDINIKTGTTDIGSNTTVKTGDLVTYDKENGMHKKVFYSFIDDKN |
| | L₂-Sac7e-L₂ | HNKKLLVIRTKGTIAGQYRVYSEEGANKSGLAWPSAFKVQLQLPDNE |
| | | VAQISDYYPRNSIDTKEYMSTLTYGFNGNVTGDDGGGGSGGGGSMA |
| | | KVRFKYKGEEKEVDTSKIKKVWRVGKMVSFTYDDNGKTGRGAVSE |
| | | KDAPKELMDMLARAEKKKGGGGSGGGGSKIGGLIGANVSIGHTLKY |
| | | VQPDFKTILESPTDKKVGWKVIFNNMVNQNWGPYDRDSWNPVYGN |
| | | QLFMKTRNGSMKAADNFLDPNKASSLLSSGFSPDFATVITMDRKASK |
| | | QQTNIDVIYERVRDDYQLHWTSTNWKGTNTKDKWTDRSSERYKID |
| | | WEKEEMTN |
| 20 | hemolysin with | MDSDINIKTGTTDIGSNTTVKTGDLVTYDKENGMHKKVFYSFIDDKN |
| | L₂-SNAP-L₂ | HNKKLLVIRTKGTIAGQYRVYSEEGANKSGLAWPSAFKVQLQLPDNE |
| | | VAQISDYYPRNSIDTKEYMSTLTYGFNGNVTGDDGGGGSGGGGSMD |
| | | KDCEMKRTTLDSPLGKLELSGCEQGLHRIIFLGKGTSAADAVEVPAP |
| | | AAVLGGPEPLMQATAWLNAYFHQPEAIEEFPVPALHHPVFQQESFTR |
| | | QVLWKLLKVVKFGEVISYSHLAALAGNPAATAAVKTALSGNPVPILI |
| | | PCHRVVQGDLDVGGYEGGLAVKEWLLAHEGHRLGKPGLGGGGGSG |
| | | GGGSKIGGLIGANVSIGHTLKYVQPDFKTILESPTDKKVGWKVIFNNM |
| | | VNQNWGPYDRDSWNPVYGNQLFMKTRNGSMKAADNFLDPNKASS |
| | | LLSSGFSPDFATVITMDRKASKQQTNIDVIYERVRDDYQLHWTSTNW |
| | | KGTNTKDKWTDRSSERYKIDWEKEEMTN |
| 21 | linker | GGGGS |
| 22 | linker | GGGGSGGGGS |
| 23 | linker | GGGGSGGGGSGGGGS |
| 24 | L-6XHis-L | GGGGSHHHHHHGGGGS |
| 25 | L₂-6XHis-L₂ | GGGGSGGGGSHHHHHHGGGGSGGGGS |
| 26 | L₃-6XHis-L₃ | GGGGSGGGGSGGGGSHHHHHHGGGGSGGGGSGGGGS |
| 27 | L-Somatostatin-L | GGGGSAGCKNFFWKTFTSCGGGGS |
| 28 | L₂-GLP1-L₂ | GGGGSGGGGSHAEGTFTSDVSSYLEGQAAKEFIAWLVKGRGGGGSGGGGS |
| 29 | L-K3-L | GGGGSKGGKGGKGGGGS |
| 30 | L-E3-L | GGGGSEGGEGGEGGGGS |
| 31 | L₂-Sac7e-L₂ | GGGGSGGGGSMAKVRFKYKGEEKEVDTSKIKKVWRVGKMVSFTYD |
| | | DNGKTGRGAVSEKDAPKELMDMLARAEKKKGGGGSGGGGS |
| 32 | L₂-SNAP-L₂ | GGGGSGGGGSMDKDCEMKRTTLDSPLGKLELSGCEQGLHRIIFLGKG |
| | | TSAADAVEVPAPAAVLGGPEPLMQATAWLNAYFHQPEAIEEFPVPAL |
| | | HHPVFQQESFTRQVLWKLLKVVKFGEVISYSHLAALAGNPAATAAV |
| | | KTALSGNPVPILIPCHRVVQGDLDVGGYEGGLAVKEWLLAHEGHRL |
| | | GKPGLGGGGGSGGGGS |
| 33 | Alpha α- | SDINIKTGTTDIGSNTTVKTGDLVTYDKENGMHKKVFYSFIDDKNHN |
| | Hemolysin | KKLLVIRTKGTIAGQYRVYSEEGANKSGLAWPSAFKVQLQLPDNEVA |
| | without Signal | QISDYYPRNSIDTKEYMSTLTYGFNGNVTGDDXXXKIGGLIGANVSIG |
| | Sequence and | HTLKYVQPDFKTILESPTDKKVGWKVIFNNMVNQNWGPYDRDSWN |
| | N-terminal | PVYGNQLFMKTRNGSMKAADNFLDPNKASSLLSSGFSPDFATVITMD |
| | Methionine | RKASKQQTNIDVIYERVRDDYQLHWTSTNWKGTNTKDKWTDRSSER |
| | | YKIDWEKEEMTN |
| 34 | N-terminal | SDINIKTGTTDIGSNTTVKTGDLVTYDKENGMHKKVFYSFIDDKNHN |
| | domain of | KKLLVIRTKGTIAGQYRVYSEEGANKSGLAWPSAFKVQLQLPDNEVA |
| | Alpha α- | QISDYYPRNSIDTKEYMSTLTYGFNGNVTGDD |
| | Hemolysin | |
| 35 | Truncated N- | DLVTYDKENGMHKKVFYSFIDDKNHNKKLLVIRTKGTIAGQYRVYS |
| | terminal | EEGANKSGLAWPSAFKVQLQLPDNEVAQISDYYPRNSIDTKEYMSTL |
| | domain of | TYGFNGNVTGDD |
| | Alpha α- | |
| | Hemolysin | |
| 36 | C-terminal | KIGGLIGANVSIGHTLKYQPDFKTILESPTDKKVGWKVIFNNMVNQN |
| | domain of | WGPYDRDSWNPVYGNQLFMKTRNGSMKAADNFLDPNKASSLLSSG |
| | Alpha α- | FSPDFATVITMDRKASKQQTNIDVIYERVRDDYQLHWTSTNWKGTN |
| | Hemolysin | TKDKWTDRSSERYKIDWEKEEMTN |

1. An α-Hemolysin polypeptide comprising an amino acid insert sequence between amino acids corresponding to amino acid positions D128 and K131 of SEQ ID NO:3, wherein the amino acid insert sequence is 5 to 70 amino acids in length.

2. The α-Hemolysin polypeptide of claim 1, wherein the α-Hemolysin polypeptide without said amino acid insert sequence has at least 80%, 85%, 90%, 91%, 92%, 93%, 94%, 95%, 96%, 97%, 98%, or 99% sequence identity to SEQ ID NO: 3.

3. The α-Hemolysin polypeptide of claim 1, wherein the amino acid sequence between amino acids corresponding to amino acid positions D128 and K131 of SEQ ID NO:3 comprises an amino acid deletion.

4. The α-Hemolysin polypeptide of claim 1, wherein the α-Hemolysin polypeptide comprises a cysteine mutation corresponding to amino acid position 130 of SEQ ID NO:3.

5. The α-Hemolysin polypeptide of claim 1, wherein the α-Hemolysin polypeptide comprises a histidine to leucine mutation corresponding to amino acid position H48 of SEQ ID NO:3.

6. The α-Hemolysin polypeptide of claim 1, wherein the α-Hemolysin polypeptide comprises a deletion corresponding to amino acid positions D2-G23 of SEQ ID NO:3.

7. The α-Hemolysin polypeptide of claim 1, wherein the amino acid insert sequence is less than 41 amino acids in length.

8. The α-Hemolysin polypeptide of claim 1, wherein the amino acid insert sequence is less than 52 amino acids in length.

9. The α-Hemolysin polypeptide of claim 1, wherein the amino acid insert sequence comprises a cysteine.

10. The α-Hemolysin polypeptide of claim 1, wherein the amino acid insert sequence comprises a glycine linker.

11. The α-Hemolysin polypeptide of claim 1, additionally comprising a bioconjugate reactive moiety.

12. The α-Hemolysin polypeptide of claim 11, wherein the bioconjugate reactive moiety is attached to an amino acid of the amino acid insert sequence.

13. The α-Hemolysin polypeptide of claim 1, additionally comprising a cargo molecule.

14. The α-Hemolysin polypeptide of claim 13, wherein the cargo molecule is attached to the amino acid insert sequence.

15. The α-Hemolysin polypeptide of claim 13, wherein the cargo molecule is covalently attached to a cysteine in the amino acid insert sequence.

16. The α-Hemolysin polypeptide of claim 13, wherein the cargo molecule is covalently attached by a bioconjugate to a cysteine in the amino acid insert sequence.

17. The α-Hemolysin polypeptide of claim 13, wherein the cargo molecule is selected from the group consisting of a polynucleotide, a polypeptide, a lipid, a carbohydrate, and a small molecule.

18. A phospholipid membrane comprising the α-Hemolysin polypeptide of claim 1.

19.-26. (canceled)

27. A peptide of the formula: 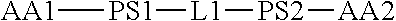
wherein
PS1 is an amino acid sequence having at least 90% sequence identity to SEQ ID: 35;
PS2 is an amino acid sequence having at least 90% sequence identity to SEQ ID: 36;
L1 is a peptidyl linker having 1-70 amino acids; and
AA1 and AA2 are independently hydrogen or amino acid sequences having 1-100 amino acids.

28.-42. (canceled)
